(19) United States
(12) Patent Application Publication
(10) Pub. No.: US 2017/0191035 A1
(43) Pub. Date: Jul. 6, 2017

(54) Ex Vivo Browning of Adipose Tissue Therapy for Reversal of Obesity and Type II Diabetes

(71) Applicant: The Trustees of Columbia Univ. in the City of New York, New York, NY (US)

(72) Inventors: Samuel K. SIA, New York, NY (US); Brian M. GILLETTE, Bronx, NY (US)

(73) Assignee: The Trustees of Columbia University in the City of New York, New York, NY (US)

(21) Appl. No.: 15/325,042

(22) PCT Filed: Jul. 10, 2015
(86) PCT No.: PCT/US15/39917

§ 371 Date: Jan. 9, 2017

Related U.S. Application Data
(60) Provisional application No. 62/023,171, filed on Jul. 10, 2014.

(51) Int. Cl. C12N 5/077; A61K 35/35

(52) U.S. Cl. CPC C12N 5/0653; A61K 35/35; C12N 2523/00; C12N 2501/39; C12N 2501/33; C12N 2501/999; C12N 2501/155; C12N 2501/119; C12N 2501/165; C12N 2501/115

(57) ABSTRACT

Provided are methods, apparatus, pharmaceutical compositions, and kits for treatment of a metabolic condition, including obesity and type 2 diabetes, by administration to a subject of a therapeutically effective amount of a cell or tissue preparation such as brown adipose microtissues or brown adipose tissue directly converted from white adipose tissue. Modified approaches to creating brown adipose tissue involve differentiation of explanted white adipose tissue and direct browning of white adipose tissue in a bioreactor rather than isolation and expansion of adipose stems cells or endothelial cells and formation and differentiation of 3D cell aggregates.

### CROSS REFERENCE TO RELATED APPLICATIONS

This application claims priority to U.S. application Ser. No. 62/023,171, filed Jul. 10, 2014, which is herein incorporated by reference in its entirety.

### STATEMENT OF GOVERNMENTAL INTEREST

This invention was made with Government support under Contract No. R01HL095477 awarded by the National Institutes of Health. The Government has certain rights in the invention.

### BACKGROUND

During the past 20 years, there has been a dramatic increase in obesity in the United States and these rates remain high. In 2010, no state had a prevalence of obesity less than 20%. Not only does obesity threaten the health of a significant portion of the U.S. population, but this crisis imposes a considerable financial burden as well. There is no shortage of research in the United States in an effort to combat obesity and obesity-related diseases.

Brown adipose tissue (BAT) is a highly metabolic form of fat tissue that natively exists in humans and other mammals. The primary function of BAT is to convert chemical energy to heat through a highly metabolic process of uncoupled respiration (thermogenesis), which is performed by numerous mitochondria containing uncoupled protein 1 (UCP1) in brown adipose cells. Until recently it was thought that adult humans lack functional BAT; however, new studies have revealed that some adults have significant amounts of active BAT which may contribute to energy expenditure and maintenance of a lean, non-diabetic phenotype. It was found that adult humans with higher amounts of brown adipose tissue tend not to be overweight or obese, and that BAT levels and activity are negatively correlated with body mass index (BMI) and body fat. The amount of BAT present in humans correlates strongly with lower body fat levels and healthy metabolism. Further, it has been found in humans that the amount of active BAT decreases with age, providing a potential link between BAT loss and age-related weight gain.

BAT's mechanism of action is primarily a function of its numerous and large mitochondria, which contain uncoupling protein 1 (UCP-1). Due to the naturally high metabolic rate of BAT (which can account for up to 20% of daily energy expenditure), BAT has great potential as an anti-obesity therapy if the amount and/or activity of BAT can be increased in humans. Adult humans and mice have brown-like or “beige” adipocytes present in white adipose deposits which are normally quiescent, but can become highly thermogenic upon appropriate stimulation. In mice, chronic stimulation through cold exposure or beta-3 adrenergic stimulation increases the extent and activity of BAT-like cells in white fat deposits, a process often called “browning.” Increasing or activating brown or “beige” adipose tissues has been shown to reduce weight and symptoms of diabetes in mouse models. See, for example, Bostrom et al., Nature 481, 463-468, 26 Jan. 2012.

Most current treatments for obesity induce weight loss by reducing caloric intake. However, it has been posed that humans naturally compensate for reduced energy intake by lowering metabolic rate, ultimately limiting the efficacy of such therapies. Other therapies for weight loss and type 2 diabetes (such as bariatric surgery and pharmaceuticals) have had limited success and exhibit numerous side effects and complications. The epidemic of obesity and diabetes, with the additional related complications of heart disease and cancer, present major public health concerns in terms of population health and medical expenses. There is a need for treating and preventing obesity and diabetes symptoms in humans that will potentially have a major impact in reducing the poor health and high costs associated with obesity, diabetes, and associated comorbidities. Harnessing BAT's capacity for increasing energy consumption via thermogenesis provides a therapy that induces weight loss by increasing metabolic rate, rather than limiting absorption of calories and nutrients. Increasing BAT levels in obese patients to similar levels as those of lean individuals provides the same benefits for reducing body mass and metabolic health in obese individuals, but with enhanced safety and efficacy compared to drugs or bariatric surgery.

### SUMMARY

The disclosure is based, at least in part, on the discovery that brown adipose microtissues (BAMs) and brown adipose tissue (BAT) injected into patients integrate with the vascular supply and burn calories stored and consumed by that patient, thereby causing weight loss.

In a first embodiment, methods are provided for isolating stem cells and endothelial cells from a subject. This is accomplished by first expanding the stem cells (e.g., that are in a range in number from 20 to 5000) and endothelial cells on a culture surface and then removing the stem cells and endothelial cells from the culture surface and mixing them together forming a cell suspension. Next, the cell suspension is placed on a non-adhesive array and cultured in a medium comprising differentiation factors that induce the stem cells to form a particular differentiated cell until a 3D aggregate of the particular differentiated cells and the endothelial cells forms on the non-adhesive array. 3D aggregates in size from about 50 to 1000 microns may be made in the method of this first embodiment using stem cells that are ASCs. The 3D aggregate may include differentiated cells that are BAT and the differentiation factors induce the formation of the BAT. These particular 3D aggregates that are made may include cells where 0-95% of the cells are ECs and 5-100% of the cells are differentiated cells (e.g., BAT cells). The 3D aggregate can include ECs concentrated to the middle of the 3D aggregate as well as particular differentiated cells concentrated on the outside of the 3D aggregate.

In a second embodiment, methods are provided for treatment for a metabolic disorder (e.g., obesity, overweight, type 2 diabetes, metabolic syndrome, impaired glucose tolerance, insulin-resistance, dyslipidemia, cardiovascular disease, and hypertension). In this method, stem cells (e.g., ASCs) and endothelial cells are isolated from a subject that is in need of treatment of the metabolic disorder. The stem cells and endothelial cells are then expanded on a culture surface (e.g., a 2D culture surface). The stem cells and endothelial cells are removed from the culture surface and then mixed together to form a cell suspension. Next, the cell suspension is placed on a non-adhesive array such as an alginate hydrogel-based microwell. The cell suspension is cultured in a medium comprising differentiation factors that induce the stem cells to form BAT until a 3D aggregate of the brown adipose tissue cells and the endothelial cells forms on the array. The 3D aggregate is in size from about 50 to 1000 microns and is then cultured in a medium containing angiogenic factors (e.g., VEGF, bFGF) until a vascularized BAM is formed. Culturing with these factors occurs so that functional markers of brown adipose thermogenesis, including uncoupled protein 1 (UCP1) and β3 adrenergic receptors (β3AR) are expressed. The vascularized BAM is recovered from the non-adhesive array. Finally, a therapeutically effective amount of the isolated vascularized BAM is administered to the subject. In this particular embodiment, the number of cells on the array is from about 10⁵ to about 10⁹ cells. Furthermore, the number of cells in the 3D aggregate is from about 50 to about 5000. Differentiation factors may be selected from the group consisting of: dexamethasone, indomethacin, insulin, and triiodothyronine (T3) and can further comprise dexamethasone, indomethacin, insulin, isobutyl-methylxanthine (IBMX), rosiglitazone, sodium ascorbate, triiodothyronine (T3), and CL316,243. A particular differentiation cocktail may be used including 50 μg/mL of sodium ascorbate, 0.85 μM insulin, 1 μM dexamethasone, 0.5 mM IBMX, 50 μM indomethacin, 250 nM T₃, 1 μM rosiglitazone, and 0 or 1 μM CL316,243. Differentiation of the stem cells can occur from about 2 days to about 3 weeks, preferably 3 weeks. In this embodiment, the vascularized BAMs are administered by injection in a therapeutically effective amount that is in a range from about 10 g to about 1 kg. The subject is preferably human.

In a third embodiment, a method of treatment for a metabolic disorder (e.g., obesity, overweight, type 2 diabetes, metabolic syndrome, impaired glucose tolerance, insulin-resistance, dyslipidemia, cardiovascular disease, and hypertension) is provided by isolating (e.g., by liposuction or surgical excision) white adipose tissue (“WAT”) from a subject. The WAT is reduced into smaller fragments by mechanical means such as mincing or dicing and cultured (e.g., in a bioreactor or culture dish) in the presence of factors (e.g., dexamethasone, indomethacin, insulin, isobutylmethylxanthine [IBMX], rosiglitazone, sodium ascorbate, triiodothyronine [T3], and CL316,243) that promote BAT differentiation, to create brown adipose-like cells. These brown adipose-like cells in clumps or clusters are then isolated and administered in a therapeutically effective amount to a subject. In certain embodiments, a differentiation factor cocktail may include 50 μg/mL of sodium ascorbate, 0.85 μM insulin, 1 μM dexamethasone, 0.5 mM IBMX, 50 μM indomethacin, 250 nM T₃, 1 μM rosiglitazone, and 0 or 1 μM CL316,243. Differentiation may occur in certain embodiments from about 2 to about 60 days, preferably 17 days, and occurs so that functional markers of brown adipose thermogenesis, including uncoupled protein 1 (UCP1) and β3 adrenergic receptors (β3AR), are expressed.

In yet another embodiment, methods further comprise assembling the aggregates of microtis sues (e.g., BAMs) or in the alternative aggregates of WAT fragments together by collecting and placing together the microtissues or WAT fragments in larger arrays (such as microwells or microchannels) of controlled shape (e.g., circular, rod, or fiber) and culturing the microtissues or WAT fragments together in the larger arrays of controlled shape in the presence of factors which promote vascularization, thereby allowing for more extensive development of connected vasculature throughout the microtis sues or WAT and prior to administering the BAT to a subject.

In certain embodiments, methods are provided for directly converting white adipose tissue to brown adipose tissue. In an aspect, this conversion takes place in one step, two steps, or three steps. In another aspect, this conversion takes place in no more than one step, no more than two steps, or no more than three steps. In an aspect, this is accomplished by first harvesting subcutaneous white adipose tissue from a subject. Then, the white adipose tissue fragments are transferred into a bioreactor. Next, the white adipose tissue fragments are cultured in the bioreactor and then exposed to culture conditions, either in the presence of media comprising browning factors (e.g., norepinephrine or those listed in Table 1), or exposed to cold temperatures in a range from about 10° C. to about 40° C., about 15° C. to about 35° C., about 20° C. to about 35° C., about 25° C. to about 35° C., about 30° C. to about 35° C., about 15° C., about 20° C., about 25° C. to about 30° C., about 25° C., about 30° C., or about 35° C. or exposed to a combination of both browning factors and cold temperature to induce conversion of white adipose tissue fragments to brown adipose tissue fragments.

In an alternative embodiment, a method of treatment for a metabolic disorder (e.g., obesity, overweight, type 2 diabetes, metabolic syndrome, impaired glucose tolerance, insulin resistance, dyslipidemia, cardiovascular disease, and hypertension) is provided by directly isolating (e.g., by liposuction or surgical excision) white adipose tissue from a subject. This is accomplished by first harvesting subcutaneous white adipose tissue from a subject. Then, the white adipose tissue fragments are transferred into a bioreactor. Next, the white adipose tissue fragments are cultured in the bioreactor in culture conditions, either in the presence of media comprising browning factors (e.g., norepinephrine or those listed in Table 1), or exposed to cold temperatures in a range from about 10° C. to about 40° C., about 15° C. to about 35° C., about 20° C. to about 35° C., about 25° C. to about 35° C., about 30° C. to about 35° C., about 15° C., about 20° C., about 25° C. to about 30° C., about 25° C., about 30° C., or about 35° C., or exposed to a combination of both browning factors and cold temperature to induce conversion of white adipose tissue fragments to brown adipose tissue fragments. The brown adipose tissue fragments are recovered from the bioreactor and administered in a therapeutically effective amount between 0.02-20 kilograms to the subject.

In a seventh embodiment, a device is provided for the collection and packing together of microtissues (e.g., BAM) from solution comprising a particle collection channel, a set of filtering channels that allows for flow of media but not the flow of particles above a given size, and an outlet channel that allows for flow of media out of the device; so that a solution containing microtissues flows through the device, trapping the microtissues in the particle collection channel while allowing media to flow around the microtis sues through the filtering channels to allow for extended culturing, thereby creating aggregated microtissues that can be directly administered to a subject.

In an eighth set of embodiments, an apparatus is provided, for example to use for ex vivo browning of WAT fragments to convert WAT to BAT. The apparatus includes a gas permeable membrane configured to enclose, at least in part, a culture chamber. The apparatus also includes a first port in fluid communication with the culture chamber, and a different second port in fluid communication with the culture chamber. The apparatus also includes a tissue access port configured to pass a tissue fragment from about 1 mm in size to about 10 mm in size into and out of the culture chamber. In some of these embodiments, the apparatus includes a rigid housing configured to hold the gas permeable membrane in a predetermined shape when the culture chamber is filled with a fluid, wherein the housing includes a vent configured to allow gas outside the housing to contact the gas-permeable membrane.

In some of these embodiments, a system includes the apparatus and the external supply for the fluid medium, and a pump configured to cause fluid to flow through the first port into the culture chamber from the supply.

In some of these embodiments, a system includes the apparatus and an environmental chamber configured to provide gas and temperature conducive to culturing tissue in the culture chamber.

Thus, in some of these embodiments, the system comprises a single-use cartridge including a gas permeable perfusion culture chamber, a prefilled media bag, a wash reagent bag, and a waste reservoir pump.

In a ninth embodiment, a pharmaceutical composition, comprising therapeutically effective amounts of a microtissue (e.g., BAMs) or BAT fragments and kits comprising them, are provided.

In another embodiment, a medium is provided that comprises browning factors and analogs thereof selected from the group consisting of insulin, hydrocortisone, and norepinephrine, and analogs thereof. Other browning factors could include dexamethasone, indomethacin, isobutylmethylxanthine (IBMX), rosiglitazone, sodium ascorbate, triiodothyronine (T3), CL316,243, retinoic acid, vascular endothelial growth factor (VEGF), basic fibroblast growth factor (bFGF), fibroblast growth factor 21 (FGF21), bone morphogenetic protein 7 (BMP7), orexin, irisin, meteorin-like, f3-aminoisobutyric acid, brain derived neurotrophic factor (BDNF), TLQP-21, leptin, capsaicin, fucoxanthin, 2-hydroxyoleic acid, conjugated linoleic acid, bofutsushosan, resveratrol, and analogs thereof. Additional browning factors could include the following classes of compounds: beta adrenergic agonists, prostaglandins, peroxisome proliferator-activated receptor gamma (PPARγ) ligands, peroxisome proliferator-activated receptor alpha (PPARζ) ligands, retinoids, thyroid hormones, AMP-activated protein kinase (AMPK) activators, n-3 fatty acids of marine origin, scallop shell powder, salmon protein hydrolysate, and analogs thereof.

Finally, a method is provided in a eleventh embodiment for identifying a subject having or at risk of developing a disorder selected from the group consisting of type 2 diabetes, metabolic syndrome, obesity or obesity-related disease, and administering to the subject a therapeutically effective amount of a BAM for treating or preventing the disorder

### BRIEF DESCRIPTION OF THE FIGURES

The following figures form part of the present specification and are included to further demonstrate certain embodiments of the present invention. The invention may be better understood by reference to one or more of these drawings in combination with the detailed description of specific embodiments presented herein.

FIG. 1. Schematic of iBAMs Assembly Process, according to an embodiment. Step (1): isolation of stem cells. Step (2): expansion of ASC and EC. Step (3): formation and development of BAMs in 3D culture. Step (4): recovery and injection. Step (5): Integration and vascularization of BAMs in vivo.

FIG. 2. Explanted mouse WAT cultured in vitro in the presence or absence of brown adipogenic and angiogenic factors, according to an embodiment. In WAT cultured in our brown adipogenic cocktail, small cells morphologically resembling brown adipocytes (containing multilocular lipid droplets) are seen interspersed within large unilocular white adipocytes (arrows point to some BAT-like cells). In panel (2A), the tissue was treated with a cocktail containing Dexamethasone, Indomethacin, Insulin, Isobutylmethylxanthine (IBMX), Rosiglitazone, Sodium Ascorbate, Triiodothyronine (T3), and CL316,243. In panel (2B), the tissue was treated with the same media as in 2A supplemented with additional angiogenic factors (VEGF and bFGF). In panel (2C), control growth media was used, and BAT-like cells are not observed. All images were taken after 17 days of culture in each condition.

FIG. 3. Images of in vitro development, according to various embodiments. Left: human adipose stem cells (ASC, unlabeled) and human GFP expressing endothelial cells (EC, green) 1 day following seeding on the hydrogel microwell array. EC are observed to migrate to the center of the cellular aggregates. Center: ASC-EC aggregates shown after several weeks in culture with factors promoting brown adipose differentiation. Lipid-containing ASC derived cells are observed around a solid core of EC. Right: ASC-EC aggregates further treated with angiogenic factors. EC are observed to form primitive blood vessel structures with open lumens, with some branching structures visible. All microwell diameters shown are 200 μm.

FIG. 4. After in vitro assembly and culture of human BAMs, the BAMs were collected and injected into a dorsal skinfold window chamber in SCID mice, according to an embodiment. Shown are a cluster of BAMs injected in a SCID mouse (48 h post implantation), with bright white showing the GFP-expressing human endothelial cells.

FIG. 5. Shown are a cluster of BAMs injected in a SCID mouse (48 h post implantation), showing the GFP-expressing human endothelial cells (EC). Some branching EC structures with open lumens are visible. Some lipid droplets in the surrounding unlabeled differentiated ASC can also be seen.

FIG. 6. After 1 week in vivo, extensive vascular networks lined with human derived (GFP expressing) EC are visibly filled with blood, according to an embodiment. The top left panel shows GFP fluorescence (human EC), top right panel shows bright field (blood filled vessels appear dark), and the bottom left panel is a merged fluorescent/bright field image. The bottom right panel is a color stereoscope image showing ectopic blood vessel formation by human EC in the mouse dorsal skinfold window chamber.

FIG. 7. After 12 days in vivo, human vascular networks are observed to continue to grow, remodel and mature, according to an embodiment. Host blood vessels are observed to grow and connect with human implant derived vessels. The top left panel shows GFP fluorescence (human EC), the top right panel shows bright field (blood filled vessels appear dark), and the bottom left panel is a merged fluorescent/bright field image. The bottom right panel is a color stereoscope image showing ectopic blood vessel formation by human EC in the mouse dorsal skinfold window chamber.

FIG. 8. Bright field and fluorescence images of ASC cultured in brown adipogenic media for 3 weeks, according to an embodiment. UCP1 immunostaining shows increasing amounts of UCP1 protein over 3 weeks' culture.

FIG. 9. This plot shows quantification of UCP1 immunostaining over the course of three weeks' differentiation, according to an embodiment. The results show an increase in UCP1 immunostaining intensity from 1-3 weeks' culture in brown adipogenic media. Medium 1 is the brown adipogenic medium and medium 2 was additionally supplemented with CL316,243.

FIG. 10. Bright field (top) and fluorescence (bottom) images showing ASC grown in brown adipogenic cocktail, according to an embodiment. In the left panel, positive immunostaining with anti-β3 adrenoreceptor antibodies indicates differentiated cells express β3AR. In the right panel, a fluorescent lipid stain highlights multilocular lipid droplets characteristic of brown adipose cells.

FIG. 11. Fluorescence images showing GFP-expressing human EC in 4 adjacent BAMs merge vascular structures after 24 h culture in media containing angiogenic factors (e.g., VEGF, bFGF), according to an embodiment.

FIG. 12. A device for the collection and packing together of microtis sues from solution that allows for direct injection, according to an embodiment.

FIG. 13. A device for the collection and packing together of microtis sues from solution that allows for direct injection wherein the PDMS collection channel and filter channel are made as separate components and then aligned on top of each other in a plastic housing, according to an embodiment.

FIG. 14A is a block diagram that illustrates an example bioreactor system for browning adipose tissue, according to an embodiment.

FIG. 14B is a photograph of bioreactor device for ex vivo browning of WAT fragments to convert WAT to BAT in the presence of browning media, according to an embodiment.

FIG. 15A are photographs of images of UCP1 staining for BAT cells following in vivo reimplantation of exBAT in mice in browning medium at 3 weeks in vitro and 3 weeks in vivo, according to an embodiment.

FIG. 15B are photographs of images of UCP1 staining for BAT cells following in vivo reimplantation of exBAT in mice in control medium at 3 weeks in vitro and 3 weeks in vivo, according to an embodiment that illustrates example viability imaging of live whole fragments at 3 weeks' culture in browning media and control media.

FIG. 16 includes photographs of images (A) of cultured WAT fragments in browning media and control media, according to an embodiment; (B) viability imaging of live whole fragments at 3 weeks' culture in browning media and control media; and (C) immunostaining of exBAT fragments to verify BAT phenotype, according to various embodiments.

FIG. 17A through FIG. 17F are diagrams that illustrate example variations in devices for automated point-of-care culturing, according to various embodiments.

FIG. 18 is a schematic illustrating oxygen consumption measurements and factors added to determine different components of respiration to monitor progress of culture, according to an embodiment.

FIG. 19 (A) is a schematic of a 3-step process for increasing brown adipose tissue (BAT) through ex vivo browning: 1) subcutaneous white adipose tissue (WAT) is harvested by liposuction or excision and cultured as tissue fragments; 2) WAT fragments are exposed to chronic browning stimuli (such as browning factors in the media) to convert the WAT to BAT; 3) the converted BAT fragments are then autologously reimplanted within WAT; (B) is a schematic of an experimental design for studies of ex vivo browning in mice: 1) subcutaneous WAT from the left inguinal depot is excised and minced into ˜5 mm fragments; 2) WAT fragments are cultured in media with and without browning factors; 3) fragments are reimplanted subcutaneously adjacent to the right inguinal WAT depot (3a) and also processed for tissue analysis in vitro (3b); (C) sets forth macroscopic images of interscapular BAT, inguinal WAT, and WAT fragments that were cultured for three weeks in media with and without browning factors, before (pre) and after 8 weeks reimplantation (post). Scale bar is 10 mm; (D) sets forth live-cell and mitochondrial staining of WAT fragments immediately after harvest (left) and after one week of culture with browning factors (right). Epifluorescence images show staining for Calcein AM (green indicates cytoplasm in live cells, designated as “I”), Mitotracker (red, designated as “II,” indicates mitochondria), and Hoescht (blue, designated as “III,” indicates nuclei). Scale bars are 150 lam (top row) and 30 μm (middle and bottom rows).

FIG. 20 sets forth confocal microscopy of ex vivo WAT to BAT conversion; (A) WAT fragments cultured in browning media for 1-3 weeks and control media for 3 weeks; (B) Mouse interscapular BAT and inguinal WAT tissue fragments. Red (designated as “II”) indicates uncoupled protein 1 (UCP1) immunostaining, green (designated as “I”) indicates lipid droplets stained with LipidTox, and blue (designated as “III”) indicates cell nuclei stained with Sytox. Scalebars are 50 μm.

FIG. 21 sets forth Confocal microscopy of BAT phenotype stability after reimplantation; (A) WAT fragments cultured in browning media for 1-3 weeks and control media for 3 weeks (images of 1-2 week), 8 weeks after reimplantation. Scale bars are 50 μm; (B) high magnification images showing channel networks of putative capillaries within explanted tissues. Scale bars are 30 μm. Red (designated as “II”) indicates UCP1 immunostaining, green (designated as “I”) indicates lipid droplets stained with LipidTox, blue (designated as “III”) indicates cell nuclei stained with Sytox, and greyscale diplays transmitted light.

FIG. 22 sets forth measurements of WAT to BAT conversion and stability after reimplantation; (A) UCP1 average intensity measurements from wide-area epifluorescence images; (B) UCP1 volume fraction; (C) lipid fraction, and (D) cell density, as measured from 3D confocal images. Error bars indicate SEM. Different numbers of asterisks indicate significant differences (p<0.001) as determined by two-way ANOVA and Bonferroni post hoc tests.

FIG. 23 sets forth Ex vivo browning of human subcutaneous WAT; (A) Confocal images of tissue fragments cultured in browning or control media and at 30° C. or 37° C. for 1 week. Scale bars are 50 μm; (B) Quantitation of UCP1 intensity, UCP1 volume fraction, lipid fraction, and cell density. Error bars indicate SEM. Different numbers of asterisks indicate statistical differences (p<0.0001) as determined by one-way ANOVA followed by Tukey post hoc tests.

FIG. 24 sets forth weight and food consumption data; (A) average daily food consumption before and after reimplantation for tissues cultured in browning and control media; (B) Maximum percentage weight loss following reimplantation, relative to weight at reimplantation; (C) Weekly percent change in weight relative to weight at time of first surgical procedure to harvest WAT. Error bars indicate SEM.

FIG. 25 sets forth an example overview of a thermograft process.

FIG. 26 sets forth a schematic design of a thermograft single-use cartridge/bioreactor system; with i) syringe used to inject WAT and collect converted BAT; ii) a gas permeable cell culture bag; iii) a peristaltic pump used to control flow rate through the chamber; iv) a culture media with browning factors; v) a pump used to control fresh media/waste exchange rate; and vi) a waste bag.

FIG. 27 sets forth an example process for ex vivo browning of adipose tissues and studies in mice. A) Illustration of experimental design for studies of ex vivo browning in mice: 1) subcutaneous WAT from the left inguinal depot is excised and minced into ˜5 mm fragments; 2) WAT fragments are cultured in media with and without browning factors; 3) fragments are reimplanted subcutaneously adjacent to the right inguinal WAT depot. B) Macroscopic images of interscapular BAT, inguinal WAT, and WAT fragments that were cultured for three weeks in media with and without browning factors, before and after 8 weeks reimplantation. Scale bar is 10 mm.

FIG. 28 sets forth a vascularized fat pad in situ at 8 weeks following reimplantation of converted BAT.

FIG. 29A-E sets for a confocal microscopy of ex vivo WAT to BAT conversion and stability after reimplantation. A) WAT fragments cultured in browning media and control media for 3 weeks, along with native WAT and BAT tissue controls. Red (designated as “II”) indicates uncoupled protein 1 (UCP1) immunostaining, green (designated as “I”) indicates lipid droplets stained with LipidTox, and blue (designated as “III”) indicates cell nuclei stained with Sytox. Scalebars are 50 μm. B-D) Quantitative measurement of WAT to BAT conversion and stability after reimplantation. B) UCP1 average intensity measurements from wide-area epifluorescence images. C) UCP1 volume fraction, and D) lipid fraction, as measured from 3D confocal images. Error bars indicate SEM. Different numbers of asterisks indicate significant differences (p<0.001) as determined by two-way ANOVA and Bonferroni post hoc tests. E) High magnification images showing channel networks of putative capillaries within explanted tissues. Scale bars are 30 μm.

FIG. 30 sets forth Conversion of human WAT to BAT from 1-3 weeks in browning media, compared to non-cultured WAT.

FIG. 31 sets forth quantification of UCP1 staining intensity of fragments cultured in browning media from 1-3 weeks, compared to control WAT and BAT tissues. Error bars indicate SEM. Different numbers of asterisks indicate significant differences (p<0.001) as determined by two-way ANOVA and Bonferroni post hoc tests.

### DETAILED DESCRIPTION

The present disclosure provides approaches to creating BAT that involve isolation and expansion of adipose stem cells and endothelial cells as well as formation and differentiation of 3D cell aggregates and directly differentiating WAT. Some potential advantages using explanted WAT include: (i) decreased complexity and time required to generate BAT, as the tissue components (blood vessels, ECM, innervation, stem cell niche) would remain intact; (ii) reduced risk and safety concerns from a regulatory perspective since tissue is less manipulated and 2D culture expansion is avoided; (iii) lipids in the WAT could provide nutrients for the developing BAT; (iv) significant amounts of WAT (>1 kg) can be obtained by liposuction, so it may be easier to generate sufficient BAT mass than by expansion of stem cells; and (v) the reduction in complexity could potentially allow BAT production in a self-contained system at the point of use, avoiding the need for shipping tissue to/from a centralized production lab (in some countries this would allow the process to fall outside the lines of cellular products regulated as biologics/therapeutics), potentially accelerating time to market. Both these approaches may include an additional step of pre-assembly of multiple BAMs in defined shapes (such as fibers) prior to injection in order to form more extensive vascular networks and accelerate blood perfusion post-implantation. As described herein, devices for the collection and packing together of microtis sues from solution allow for direct injection into the subject.

A cell therapy approach based on the isolation, expansion, differentiation and delivery of engineered BAT-like cells potentially faces highly complex process development, characterization, and logistical constrains that currently have high costs and limited scalability. Alternative approaches involve a novel and more direct approach for increasing BAT mass in humans through ex vivo browning of adipose tissue in a perfusion bioreactor. In this approach, subcutaneous WAT is first harvested form the patient utilizing techniques and tools of commonly performed autologous fat-transfer procedures known in the art. Next, the WAT is aseptically transferred into a perfusion bioreactor that mimics native vascular and interstitial flow and in culture conditions either in the presence of media comprising browning factors (e.g., norepinephrine or those listed in Table 1), or exposed to cold temperatures in a range from about 15° C. to 35° C. (preferably 30° C.), or exposed to a combination of both browning factors and cold temperature to induce conversion of white adipose tissue fragments to brown adipose tissue fragments to induce development of UCP1-expressing brown adipocytes. The tissue is washed to remove norepinephrine, withdrawn into a fat transfer syringe, and then reimplanted back into subcutaneous WAT.

The harvested WAT fragments are directly converted (e.g., development of UCP-1-expressing brown adipocytes) to BAT and therefore allows for a simplified approach for engineering autologous BAT tissue as compared to engineering BAT tissue from stem cells. These UCP1-positive thermogenic cells, often referred to as “beige” or “BRITE” (brown-in-white) adipocytes, develop through both transdifferentiation of existing WAT cells and by differentiation of proliferating progenitors. Browning of subcutaneous WAT fragments outside the body for autologous reimplantation adapts the already widely practiced techniques of autologous fat-transfer procedures to obtain viable subcutaneous WAT and to regraft the tissue with a high degree of survival and function. The key advantages of this approach are (i) that it is a simple process that leverages existing clinical workflow for autologous fat transfer procedures by physicians (e.g., plastic surgeons) in a short procedure in-office; (ii) scalable production at point of care while avoiding centralized tissue shipping logistics; (iii) a greatly simplified process, in comparison to isolated stem cell-based approaches, that allows for assessment of tissue viability and extent of browning; and (iv) overall minimizing of technical, safety, regulatory, logistical, and clinical implementation barriers to autologous BAT grafting in humans, including avoiding exposing the patient to extended cold exposure or adrenergic drugs.

### 1. Definitions

The following terms as used herein have the corresponding meanings given here. Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lan, Antibodies: A Laboratory Manual, Cold Spring Harbor Laboratory Press, Cold Spring Harbor, N.Y. (1990); Principles of Neural Science, 4th ed., Eric R. Kandel, James H. Schwart, Thomas M. Jessell editors. McGraw-Hill/Appleton & Lange: New York, N.Y. (2000). Unless defined otherwise, all technical and scientific terms used herein have the same meaning as commonly understood by one of ordinary skill in the art.

The terms “individual” “subject” or “patient” are used interchangeably and mean any mammalian subject for whom diagnosis, treatment, or therapy is desired, particularly humans. A “subject” as used herein generally refers to any living multicellular organism. Subjects include, but are not limited to, plants and animals (e.g., cows, pigs, horses, sheep, dogs, and cats), including hominoids (e.g., humans, chimpanzees, and monkeys). The term includes transgenic and cloned species. The term “patient” refers to both human and veterinary subjects.

The term “administering” means “delivering in a manner which is affected or performed using any of the various methods and delivery systems known to those skilled in the art.” Administering can be performed, for example, orally, or intravenously, via implant, transmucosally, transdermally, intradermally, intramuscularly, subcutaneously, or intraperitoneally. Administering can also be performed, for example, once, a plurality of times, and/or over one or more extended periods.

The term “brown adipose tissue” or “BAT” means brown fat cells or plurivacuolar cells that are polygonal in shape and have considerable cytoplasm, with lipid droplets scattered throughout. BAT or brown fat is one of two types of fat or adipose tissue (the other being white adipose tissue) found in mammals. It is especially abundant in newborns and in hibernating mammals. Its primary function is to generate body heat in animals or newborns that do not shiver. In contrast to white adipocytes (fat cells), which contain a single lipid droplet, brown adipocytes contain numerous smaller droplets and a much higher number of (iron-containing) mitochondria, which make it brown. White adipose tissue (WAT) means white fat cells or monovacuolar cells that contain a large lipid droplet surrounded by a layer of cytoplasm, the nucleus of which is flattened and located on the periphery. Brown fat also contains more capillaries than white fat, since it has a greater need for oxygen than most tissues.

The term “brown adipose microtissue” or “BAM,” as used herein, means a native-like development of small clusters of thermogenic brown fat-like cells that can incorporate blood vessels, which are small in size (about 50 to 1000 microns in diameter, 1 micron, μm, =10⁻⁶ meters).

The term “white adipose tissue” as used herein, “WAT,” is one of the two types of adipose tissue found in mammals. The other kind of adipose tissue is brown adipose tissue. In healthy, non-overweight humans, white adipose tissue composes as much as 20% of the body weight in men and 25% of the body weight in women. Its cells contain a single large fat droplet, which forces the nucleus into a thin rim at the periphery. They have receptors for insulin, growth hormones, norepinephrine and glucocorticoids. White adipose tissue is used as a store of energy.

The term “adipose stem cells” as used herein, “AS's,” are obtained from a patient's fat through biopsy, excision or liposuction. Stem cells from any source that can be induced to differentiate into BAT when contacted with the BAT differentiation factors described herein.

The term “endothelial cells” or “ECs” are cells that line and form blood vessels,

The term “differentiation factor” as used herein means any substance that promotes a change in phenotype and gene expression of a pluripotent stem cell to that of a further differentiated cell. Examples of differentiation factors are described herein.

The term “angiogenic factor” as used herein means any factor that promotes the physiological process through which new blood vessels form from pre-existing vessels. Examples of angiogenic factors are described herein.

The term “browning factor” as used herein means any factor that promotes the physiological process through which white adipose tissue converts to brown adipose tissue. Examples of browning factors are described herein and are listed in Table 1.

The terms “therapeutically effective amount” or “an effective amount,” or a “prophylactically effective amount,” which are used interchangeably, mean an amount sufficient to mitigate, decrease or prevent the symptoms associated with the conditions disclosed herein, including diseases associated with diabetes, metabolic syndrome, obesity, and other related conditions contemplated for therapy with the compositions of the present invention. The phrases can mean an amount sufficient to produce a therapeutic result. Generally, the therapeutic result is an objective or subjective improvement of a disease or condition, achieved by inducing or enhancing a physiological process, blocking or inhibiting a physiological process, or in general terms performing a biological function that helps in or contributes to the elimination or abatement of the disease or condition. For example, eliminating or reducing or mitigating the severity of a disease or set of one or more symptoms. The full therapeutic effect does not necessarily occur by administration of one dose and may occur only after administration of a series of doses. Thus, a therapeutically effective amount further includes an amount effective to decrease weight gain, decrease fat mass, and increase weight loss.

“Treating” a disease means taking steps to obtain beneficial or desired results, including clinical results, such as mitigating, alleviating or ameliorating one or more symptoms of a disease; diminishing the extent of disease; delaying or slowing disease progression; ameliorating and palliating or stabilizing a metric (statistic) of disease; or causing the subject to experience a reduction, delayed progression, regression or remission of the disorder and/or its symptoms. “Treatment” refers to the steps taken. In one embodiment, recurrence of the disorder and/or its symptoms is prevented. In the preferred embodiment, the subject is cured of the disorder and/or its symptoms. “Treatment” or “treating” can also refer to therapy, prevention and prophylaxis and particularly refers to the administration of medicine or the performance of medical procedures with respect to a patient, for either prophylaxis (prevention) or to cure (if possible) or reduce the extent of or likelihood of occurrence of the infirmity or malady or condition or event in the instance where the patient is afflicted. More particularly, as related to the present invention, “treatment” or “treating” is defined as the application or administration of a therapeutic agent to a patient who has a disease, a symptom of disease, or a predisposition toward development of a disease. Treatment can slow, cure, heal, alleviate, relieve, alter, mitigate, remedy, ameliorate, improve or affect the disease, a symptom of the disease or the predisposition toward disease. In the present invention, the treatments using the agents described may be provided to prevent diabetes, metabolic syndrome, and obesity or obesity-related diseases.

“Metabolic condition” or “metabolic disorder” or “metabolic syndrome” means a disease characterized by spontaneous hypertension, dyslipidemia, insulin resistance, hyperinsulinemia, increased abdominal fat and increased risk of coronary heart disease. As used herein, “metabolic condition” or “metabolic disorder” or “metabolic syndrome” shall mean a disorder that presents risk factors for the development of type 2 diabetes mellitus and cardiovascular disease and is characterized by insulin resistance and hyperinsulinemia and may be accompanied by one or more of the following: (a) glucose intolerance, (b) type 2 diabetes, (c) dyslipidemia, (d) hypertension and (e) obesity.

“Obesity” means a condition in which the body weight of a mammal exceeds medically recommended limits by at least about 20%, based upon age and skeletal size. “Obesity” is characterized by fat cell hypertrophy and hyperplasia. “Obesity” may be characterized by the presence of one or more obesity-related phenotypes, including, for example, increased body mass (as measured, for example, by body mass index, or “BMI”), altered anthropometry, basal metabolic rates, or total energy expenditure, chronic disruption of the energy balance, increased fat mass as determined, for example, by DEXA (Dexa Fat Mass percent), altered maximum oxygen use (V02), high fat oxidation, high relative resting rate, glucose resistance, hyperlipidemia, insulin resistance, and hyperglycemia. See also, e.g., Hopkinson et al. (1997) Am J Clin Nutr 65(2): 432-8 and Butte et al. (1999) Am J Clin Nutr 69(2): 299-307. “Overweight” individuals generally have a body mass index (BMI) between 25 and 30. “Obese” individuals or individuals suffering from “obesity” are generally individuals having a BMI of 30 or greater. Obesity may or may not be associated with insulin resistance.

An “obesity-related disease” or “obesity related disorder” or “obesity related condition,” which are all used interchangeably, refers to a disease, disorder, or condition, which is associated with, related to, and/or directly or indirectly caused by obesity, including coronary artery disease/cardiovascular disease, hypertension, cerebrovascular disease, stroke, peripheral vascular disease, insulin resistance, glucose intolerance, diabetes mellitus, hyperglycemia, hyperlipidemia, dyslipidemia, hypercholesteremia, hypertriglyceridemia, hyperinsulinemia, atherosclerosis, cellular proliferation and endothelial dysfunction, diabetic dyslipidemia, HIV-related lipodystrophy, peripheral vessel disease, cholesterol gallstones, cancer, menstrual abnormalities, infertility, polycystic ovaries, osteoarthritis, sleep apnea, metabolic syndrome (Syndrome X), type 2 diabetes, diabetic complications including diabetic neuropathy, nephropathy, retinopathy, cataracts, heart failure, inflammation, thrombosis, congestive heart failure, and any other cardiovascular disease related to obesity or an overweight condition and/or obesity related asthma, airway, and pulmonary disorders.

An individual “at risk” may or may not have detectable disease, and may or may not have displayed detectable disease prior to the treatment methods described herein. “At risk” denotes an individual who is determined to be more likely to develop a symptom based on conventional risk assessment methods or has one or more risk factors that correlate with development of diabetes, metabolic syndrome, or obesity or an obesity-related disease, or a disease for which BAM administration provides a therapeutic benefit. An individual having one or more of these risk factors has a higher probability of developing diabetes, metabolic syndrome, obesity, or an obesity-related disease, than an individual without these risk factors. Examples (i.e., categories) of risk groups are well known in the art and discussed herein.

A “kit” is any manufacture (e.g., a package or container) comprising at least one reagent, e.g., a medicament for treatment of a disease, or a probe for specifically detecting a biomarker gene or protein of the invention. In certain embodiments, the manufacture is promoted, distributed, or sold as a unit for performing the methods of the present invention.

As used herein, a mammal refers to human and non-human primates and other mammals including but not limited to human, mouse, rat, sheep, monkey, goat, rabbit, hamster, horse, cow, pig, cat, dog, etc.

“Non-human mammal,” as used herein, refers to any mammal that is not a human; “non-human primate” as used herein refers to any primate that is not a human.

“Stem cell” as used herein refers to an undifferentiated cell which is capable of essentially unlimited propagation either in vivo or ex vivo and capable of differentiation to other cell types. This can be differentiation to certain differentiated, committed, immature, progenitor, or mature cell types present in the tissue from which it was isolated, or dramatically differentiated cell types. In general, stem cells used to carry out the present invention are progenitor cells, and are not embryonic, or are “nonembryonic,” stem cells (i.e., are not isolated from embryo tissue). Stem cells can be “totipotent,” meaning that they can give rise to all the cells of an organism as for germ cells. Stem cells can also be “pluripotent,” meaning that they can give rise to many different cell types, but not all the cells of an organism. Stem cells can be highly motile. Stem cells are preferably of mammalian or primate origin and may be human or non-human in origin consistent with the description of animals and mammals as given above. The stem cells may be of the same or different species of origin as the subject into which the stem cells are implanted.

“Progenitor cell” as used herein refers to an undifferentiated cell that is capable of substantially or essentially unlimited propagation either in vivo or ex vivo and capable of differentiation to other cell types. Progenitor cells are different from stem cells in that progenitor cells are viewed as a cell population that is differentiated in comparison to stem cells and progenitor cells are partially committed to the types of cells or tissues which can arise therefrom. Thus progenitor cells are generally not totipotent as stem cells may be. As with stem cells, progenitor cells used to carry out the present invention are preferably nonembryonic progenitor cells. Progenitor cells are preferably of mammalian or primate origin and may be human or non-human in origin consistent with the description of animals and mammals as given above. The progenitor cells may be of the same or different species of origin as the subject into which the progenitor cells are implanted.

### 2. Overview

It has been discovered that using BAMs and direct administration of BAT prevent and treat obesity and diabetes. More specifically, a method for treatment of a metabolic condition, including obesity and type 2 diabetes, may occur by administration of a therapeutically effective amount of a cell preparation such as brown adipose microtissues to a mammal, wherein the microtissues comprise adipose stem cells and endothelial cells.

Accordingly it is determined that pharmacological agents that increase amounts of active BAT or stimulate “browning” of human white fat could be used to counter obesity and diabetes through increasing energy expenditure. However, selective expansion or activation of BAT using drugs is challenging (e.g., due to the complex nature of BAT development from progenitor cells in vivo, and activation/differentiation compounds can exhibit off-target effects). Hence, Applicants have developed a method to increase a patient's amount and activity of BAT through implantation of engineered BAT grafts that are produced in vitro.

Some embodiments include a method to produce engineered BAT grafts that can prevent or reverse the development of obesity and type 2 diabetes symptoms after implantation. Some embodiments include the engineered tissue itself.

The engineered BAT tissue recapitulates native-like structure, composition, and function of native BAT tissue.

Approaches other than isolated stem cell-based methods involve ex vivo browning of WAT fragments in a culture device such as a bioreactor wherein culturing occurs in the presence of browning factors. The direct conversion of harvested WAT fragments to BAT enables a vastly simplified approach for engineering autologous BAT tissue. By browning WAT outside the body, this direct procedure avoids exposing the patient to cold exposure or adrenergic drugs. By incorporating media and washing reagents with a closed automated perfusion system, sterility and consistency of the process is ensured while avoiding static culture and manual media changes. Such a system should be scalable and low-cost compared to a centralized cell bioprocess using autologous stem cells, while having robust process control to ensure patient safety and consistent production.

In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

### 3. Background

### Obesity and Type 2 Diabetes

According to the definition recommended by the World Health Organization (WHO) expert committee for the classification of overweight and obesity, today, close to 65% of the U.S. adult population is overweight, and among them, above 30% are obese (Flegal K M, Carroll M D, Ogden C L, Johnson C L: Prevalence and trends in obesity among US adults, 1999-2000. JAMA 2002, 288:1723-1727, which is herein incorporated by reference in its entirety). The exact molecular and cellular connection between obesity and type 2 diabetes has not been entirely explained. In particular, there is no unifying hypothesis that explains the various states of “garden-variety” insulin resistance associated with diet-induced obesity. One of the hypotheses highlights the pathological roles of lipid abnormality accompanying obesity or high body weight, and postulates that accumulation of fatty acids or fatty acid derivatives in muscle and liver produce insulin resistance (McGarry JD: Banting lecture 2001: Dysregulation of fatty acid metabolism in the etiology of type 2 diabetes. Diabetes 2002, 51:7-18; which is herein incorporated by reference in its entirety).

Epidemiological studies indicate that the development of type 2 diabetes takes place over a long period of time from the initial decline of insulin effectiveness, ultimately progressing to frank diabetes when β-cell function collapses. In most patients, insulin resistance can be detected long before the deterioration of glucose intolerance occurs. Approximately 5 to 10% of glucose-intolerant patients progress to frank type 2 diabetes in a given year. Inasmuch as metabolic syndrome emphasizes the condition of insulin resistance, the syndrome itself is not type 2 diabetes, but a large percentage of the people with metabolic syndrome will develop type 2 diabetes if the condition of insulin sensitivity is not improved.

Type 2 diabetes usually begins after the age of 40 (which accounts for its previously used name, maturity-onset diabetes). Type 2 diabetes is characterized by altered insulin secretory dynamics with retention of endogenous pancreatic insulin secretion, absence of ketosis (accounting for another of its names, ketosis-resistant diabetes), and insulin resistance due to diminished target-cell action of insulin. Although type 2 diabetes is heterogeneous, both of the major pathogenetic mechanisms (i.e., impaired islet beta-cell function [impaired insulin secretion] and impaired insulin action [insulin resistance or decreased insulin sensitivity]) are operative in variable degrees in most patients. Thus, impairments in insulin secretory response and insulin action are the result of dynamic processes that are marginally understood. There is still no cure for type 2 diabetes and treatment is at best a strategy of control. Therefore there is a great need for understanding the underlying causes of metabolic syndrome, especially of diabetes and obesity, and for animal models.

### 4. Summary of Experimental Results and Embodiments of the Invention

In an aspect, the disclosure provides for the following:
Injectable BAMs were created using isolation and expansion of adipose stem cells and endothelial cells that are induced by contact with certain differentiation factors to differentiate into BAT and form 3D cell aggregates;
The present methods for forming aggregates and microtissues or BAT from stem cells in culture can be applied to produce aggregates and microtissues or other differentiated cell types by culturing stem cells in a cocktail of differentiation factors known to produce the desired differentiated cell type;
Injectable BAMs were also created from differentiated explants of explanted white adipose tissue;
Multiple BAMs may be pre-assembled into defined shapes prior to injection in order to form more extensive vascular networks and accelerate blood perfusion post-injection;
Ex vivo “browning” of WAT fragments occurred in the presence of browning media promoting conversion of WAT to BAT; and
“Browned” adipose tissue can remain browned for at least several weeks following implantation.

### 5. Isolated Stem Cell-Based Approach

### Cell Types

Stem cells and ECs used in the present disclosure can be isolated from a variety of tissues and organs including, but not limited to, for example, adipose tissue (e.g., adipose tissue deposits), muscle tissue, bone tissue (e.g., bone marrow). Stem cells and ECs can also be derived from induced pluripotent stem cells that are created from any human or mammalian cell type. In particular embodiments for making BAT and BAM, stems cells and ECs are obtained from extracted subcutaneous WAT (“sWAT”).

BAMS made by the present methods incorporate three important facets of the native brown adipose microenvironment: tight packing of cells in a three-dimensional (3D) configuration, a supportive collagen extracellular matrix, and highly dense microvascular architecture. Tight cell-cell association and 3D arrangement in scaffold-free cell aggregates have been shown to promote enhanced differentiation and function of many cell types, including adipocytes (Wang 2009).

ASCs and ECs can be identified by determining the presence or absence of one or more cell surface expression markers. Exemplary cell surface markers that can be used to identify an ASC include ALCAM/CD166, Integrin alpha 4 beta 1, Aminopeptidase Inhibitors, Integrin alpha 4 beta 7/LPAM-1, Aminopeptidase N/ANPEP, Integrin alpha 5/CD49e, CD9, Integrin beta 1/CD29, CD44, MCAM/CD146, CD90/Thy1, Osteopontin/OPN, Endoglin/CD105, PUM2, ICAM-1/CD54, SPARC, Integrin alpha 4/CD49d, VCAM-1/CD106, and ECs include but are not limited to EC-specific marker (CD31 protein), ACE/CD143, MCAM/CD146, C1q R1/CD93, Nectin-2/CD112, VE-Cadherin, PD-ECGF/Thymidine Phosphorylase, CC Chemokine Receptor D6, Podocalyxin, CD31/PECAM-1, Podoplanin, CD34, S 1P1/EDG-1, CD36/SR-B3, S1P2/EDG, CD151, S1P3/EDG-3, CD160, S1P4/EDG-6, CD300LG/Nepmucin, S1P5/EDG-8, CL- 1/COLEC11, E-Selectin/CD62E, CL-P1/COLEC12, E-Selectin (CD62E)/P-Selectin (CD62P), Coagulation Factor III/Tissue Factor, P-Selectin/CD62P, DC-SIGNR/CD299, SLAM/CD150, DCBLD2/ESDN, Stabilin-1, EMMPRIN/CD147, Stabilin-2, Endoglin/CD105, TEM7/PLXDC1, Endomucin, TEM8/ANTXR1, Endo sialin/CD248, Thrombomodulin/BDCA-3, EPCR, THSD1, Erythropoietin R, Tie-2, ESAM, TNF RI/TNFRSF1A, FABPS/E-FABP, TNF RII/TNFRSF1B, FABP6, TRA-1-85/CD147, ICAM-1/CD54, TRAIL R1/TNFRSF10A, ICAM-2/CD102, TRAIL R2/TNFRSF10B, IL-1 RI, VCAM-1/CD106, IL-13 R alpha 1, VE-Statin, Integrin alpha 4/CD49d, VEGF R1/Flt-1, Integrin alpha 4 beta 1, VEGF R2/KDR/Flk-1, Integrin alpha 4 beta 7/LPAM-1, VEGF R3/Flt-4, Integrin beta 2/CD18, VGSQ, KLF4, vWF-A2, LYVE-1.

The cells are preferably autologous, but allogeneic or xenogeneic cells can also be used. Methods are provided for forming a 3D array by isolating stem cells and endothelial cells from a subject; expanding the stem cells (e.g., that are in a range from 20 to 5000) and endothelial cells on a culture surface; removing the stem cells and endothelial cells from the culture surface and mixing them together forming a cell suspension; placing the cell suspension on a non-adhesive array; and culturing the cell suspension in a medium comprising differentiation factors that induce the stem cells to form a particular differentiated cell until a 3D aggregate of the particular differentiated cells and the endothelial cells forms on the non-adhesive array. 3D aggregates from about 50 to 1000 microns may be made in the method of this first embodiment using stem cells that are ASCs. The 3D aggregate may include differentiated cells that are BAT and the differentiation factors induce the formation of the BAT. These particular 3D aggregates that are made may include cells where 0-95% of the cells are ECs and 5-100% of the cells are ASCs. The 3D aggregate can include ECs concentrated to the middle of the 3D aggregate and the particular differentiated cells are concentrated on the outside of the 3D aggregate. In some embodiments, the cells are allogeneic or xenogeneic; and if necessary, immune suppression can be administered to prevent rejection of the cells.

### 6. Differentiation Methods

Described herein are methods for engineering microtissues, e.g., BAMs using an isolated stem cell-based approach or direction induction of WAT. The stem cells such as ASCs, or in the alternative, fragments of WAT are induced to differentiate into BAT cells by culturing in differentiation media. ECs are co-cultured with the stem cells. As a negative control ASCs and ECs may be cultured for an equivalent period of time in mesenchymal stem cell growth media without differentiation factors. The concentration as well as the treatment time will be sufficient to increase the number of differentiated BAT cells or cells with the characteristic of mature BAT cells. Both the amount and the treatment time can be determined by one of skill in the art using known methods.

In some embodiments for making BAT, the differentiation cocktail includes, but is not limited to dexamethasone, indomethacin, insulin, isobutylmethylxanthine (IBMX), rosiglitazone, sodium ascorbate, triiodothyronine (T3), and CL316,243. The minimal exemplary differentiation cocktails for various types of differentiated cells (including BAT) include: T₃, indomethacin, dexamethasone, insulin. One of ordinary skill in the art could contemplate a vast number of differentiation factors for any numbers of different cell types are readily available in the art. For example, factors can be added to differentiate stem cells into liver cells, cardiac and skeletal muscle cells, pancreas cells, bone cells, white adipocytes, and lung cells.

In other embodiments, the methods include evaluating the level of BAT adipogenesis in the cell or cell population by measuring one or more of BAT specific markers, such as uncoupling protein 1 (UCP1), cell death-inducing DFF45-like effector A (CIDEA), PPAR gamma coactivator (PGC)-1 alpha, and/or PPAR gamma coactivator (PGC)-1 beta and/or PRDM-16, CYC1, NDUFAll, NDUFA13,CMT1A, ELOVL3, DIO2, LHX8, COX8A and/or CYFIP2; BAT morphology (using visual, e.g., microscopic, inspection of the cells); or BAT thermodynamics, e.g., cytochrome oxidase activity, Na+-K+-ATPase enzyme units, or other enzymes involved in BAT thermogenesis, uncoupled respiration (measuring cellular oxygen consumption in the presence of oligomycin, which blocks ATP synthase), metabolic rate, glucose consumption rate, and/or fatty acid oxidation rate. Characteristic markers of BAT can also expressed in other tissues. For example, beta 3 adrenergic receptor is involved in BAT thermogenesis but can also be found in other tissues such as the heart and prostate.

In some embodiments, the methods include treating cells with cyclic AMP (cAMP), or an analogue thereof, such as dibutryl cAMP, or β3-adrenergic agonist such as CL316249 to assess the ability of the cells to activate thermogenesis. Cold-induced thermogenesis in vivo is mediated through a signaling cascade involving the sympathetic nervous system and activation of the β3-adrenergic receptor in BAT. These events result in an increase of cytoplasmic cAMP levels, which then triggers expression of genes involved in thermogenesis in mature brown adipocytes. To determine if the differentiated cells become bona fide brown adipocytes, the expression of thermogenic genes, such as UCP-1, in differentiated adipocytes treated with the cell-penetrant cAMP analogue dibutyryl cAMP (Sigma) or β3-adrenergic agonist CL316249 (Sigma) can be measured. These methods include assessing (e.g., measuring) the expression of one or more genes involved in thermogenesis in mature brown adipocytes. Exemplary genes include, but are not limited to, UCP-1, CIDEA, PGC-1, PRDM16, and genes involved in mitochondrial biogenesis and function. Cells that show expression of one or more of these genes are identified as mature BAT cells and/or cells with characteristics of a mature brown adipocyte. In addition to gene expression, oxygen consumption in vitro can be measured, including uncoupled vs. coupled respiration.

In some embodiments, the methods include evaluating WAT differentiation, By evaluating a WAT specific marker, such as one or more of resistin, TCF21, leptin and/or nuclear receptor interacting protein 1 (RIP140), and/or WAT morphology. WAT and BAT can be distinguished by routine techniques, e.g., morphologic changes specific to WAT or BAT, or evaluation of WAT-specific or BAT-specific markers. For example, BAT cells can be identified by expression of uncoupling protein (UCP), e.g., UCP-1.

### 7. Methods of Treatment

Methods are provided for treatment for a metabolic disorder (e.g., obesity, overweight, type 2 diabetes, metabolic syndrome, impaired glucose tolerance, insulin-resistance, dyslipidemia, cardiovascular disease, and hypertension). In an isolated stem cell-based method, stem cells (e.g., ASCs) and endothelial cells are isolated from a subject that is in need of treatment of the metabolic disorder. The stem cells and endothelial cells are then expanded on a culture surface (e.g., a 2D culture surface). The stem cells and endothelial cells are removed from the culture surface and then mixed together to form a cell suspension. Next, the cell suspension is placed on a non-adhesive array such as an alginate hydrogel-based microwell. The cell suspension is cultured in a medium comprising differentiation factors that induce the stem cells to form brown adipose tissue until a 3D aggregate of the brown adipose tissue cells and the endothelial cells forms on the array. The non-adhesive array may be a hydrogel surface of alginate in hydrogel-based microwells. Other non-adhesive hydrogels could include but are not limited to agarose and poly-ethylene glycol (PEG)-based hydrogels. The number of cells in the 3D aggregate and the size of the aggregate can be controlled.

The 3D aggregate from about 50 to 1000 microns is then cultured in a medium containing angiogenic factors (e.g., VEGF, bFGF) until a vascularized brown adipose microtissue is formed. Angiogenic factors include, but are not limited to, Angiogenin, Angiopoietin-1, Del-1 Fibroblast growth factors: acidic (aFGF) and basic (bFGF), Follistatin, Granulocyte colony-stimulating factor (G-CSF), Hapatocyte growth factor (HGF)/scatter factor (SF), Interleukin-8 (IL-8), Leptin, Midkine, Placental growth factor, Platelet-derived endothelial cell growth factor (PD-ECGF), Platelet-derived growth factor-BB (PDGF-BB), Pleiotrophin (PTN) Proliferin, Transforming growth factor-alpha (TGF-alpha), Transforming growth factor-beta (TGF-beta), Tumor necrosis factor-alpha (TNF-alpha), Vascular endothelial growth factor (VEGF)/vascular permeability factor (VPF) until a vascularized BAM is formed; recovering the vascularized BAM from the non-adhesive array; and administering a therapeutically effective amount of the isolated vascularized BAM to the subject. Culturing with factors occurs so that functional markers of brown adipose thermogenesis, including uncoupled protein 1 (UCP1) and β3 adrenergic receptors (β3AR) are expressed. The vascularized brown adipose microtissue is recovered from the non-adhesive array.

A therapeutically effective amount of the isolated vascularized BAT is administered to the subject. In this particular embodiment, the number of cells on the array is from about 10⁵ to about 10⁹ cells. Furthermore, the number of cells in the 3D aggregate is from about 50 to about 5000. Differentiation factors may be selected from the group consisting of: dexamethasone, indomethacin, insulin, and triiodothyronine (T3) and can further comprise dexamethasone, indomethacin, insulin, isobutylmethylxanthine (IBMX), rosiglitazone, sodium ascorbate, triiodothyronine (T3), and CL316,243. A particular differentiation cocktail may be used including 50 μg/mL of sodium ascorbate, 0.85 μM insulin, 1 μM dexamethasone, 0.5 mM IBMX, 50 μM indomethacin, 250 nM T₃, 1 μM rosiglitazone, and 0 or 1 μM CL316,243. Differentiation of the stem cells can occur from about 2 days to about 3 weeks, preferably 3 weeks. In this embodiment, the vascularized BAMs are administered by injection in a therapeutically effective amount that is in a range from about 10 g-about 1kg. The subject is preferably human.

In a third embodiment, a method of treatment for a metabolic disorder (e.g., obesity, overweight, type 2 diabetes, metabolic syndrome, impaired glucose tolerance, insulin-resistance, dyslipidemia, cardiovascular disease, and hypertension) is provided by directly isolating (e.g., by liposuction or surgical excision) white adipose tissue from a subject. The white adipose tissue is reduced into smaller fragments by mechanical means such as mincing or dicing and cultured (e.g., in a bioreactor or culture dish) in the presence of factors (e.g., dexamethasone, indomethacin, insulin, isobutylmethylxanthine (IBMX), rosiglitazone, sodium ascorbate, triiodothyronine (T3), and CL316,243) that promote brown adipose tissue differentiation, to create brown adipose-like cells. These brown adipose-like cells in clumps or clusters are then isolated and administered in a therapeutically effective amount to a subject. In certain embodiments, a differentiation factor cocktail may include 50 μg/mL of sodium ascorbate, 0.85 μM insulin, 1 μM dexamethasone, 0.5 mM IBMX, 50 μM indomethacin, 250 nM T₃, 1 μM rosiglitazone, and 0 or 1 μM CL316,243. Differentiation may occur in certain embodiments from about 2 to about 60 days, preferably 17 days, and occurs so that functional markers of brown adipose thermogenesis, including uncoupled protein 1 (UCP1) and β3 adrenergic receptors (β3AR) are expressed.

In yet another embodiment, methods further comprise assembling the aggregates of microtissues (e.g., BAMs) or in the alternative aggregates of white adipose tissue fragments together by collecting and placing together the microtissues or white adipose tissue fragments in larger arrays (such as microwells or microchannels) of controlled shape (e.g., circular, rod, or fiber) and culturing the microtissues or white adipose tissue fragments together in the larger arrays of controlled shape in the presence of factors which promote vascularization, thereby allowing for more extensive development of connected vasculature throughout the microtissues prior to administering the BAM to a subject.

A method is provided for in a seventh embodiment for identifying a subject having or at risk of developing a disorder selected from the group consisting of type 2 diabetes, metabolic syndrome, obesity or obesity-related disease, and administering to the subject a therapeutically effective amount of a BAM for treating or preventing the disorder. The present methods and microtis sues can also be used to treat other disorders wherein administering vascularized microtis sues of desired differentiated cell types will be therapeutically useful. For example microtissues of osteocytes may be administered to accelerate bone growth, white adipose tissue could be administered for cosmetic/reconstructive surgeries, cardiac or skeletal muscle could be administered for cardiac or muscle disease, and pancreatic tissue could be administered to counter type 1 diabetes.

In some embodiments, the methods include identifying a subject in need of treatment (e.g., an overweight or obese subject, with a body mass index [BMI] of 25-29 or 30 or above, or a subject with a weight-related disorder) and administering to the subject an effective amount of BAMs. A subject in need of treatment with the methods described herein can be selected based on the subject's body weight or body mass index. In some embodiments, the methods include evaluating the subject for one or more of: weight, adipose tissue stores, adipose tissue morphology, insulin levels, insulin metabolism, glucose levels, thermogenic capacity, and cold sensitivity. In some embodiments, subject selection can include assessing the amount or activity of BAT in the subject and recording these observations.

In an alternative embodiment, it is possible to directly convert white adipose tissue to brown adipose tissue when subcutaneous WAT is first harvested from the patient utilizing techniques and tools of commonly performed autologous fat-transfer procedures. The UCP1-positive thermogenic BAT cells develop through both transdifferentiation of existing WAT cells and by differentiation of proliferating progenitors. The WAT is aseptically transferred into a perfusion bioreactor that mimics native vascular and interstitial flow, and then exposed to culture conditions either in the presence of media comprising browning factors (e.g., norepinephrine or those listed in Table 1), or exposed to cold temperatures in a range from about 15° C. to 35° C. (preferably 30° C.), or exposed to a combination of both browning factors and cold temperature to induce conversion of white adipose tissue fragments to brown adipose tissue fragments. A chemically defined, animal- and human-component-free medium has been developed that supports browning of WAT fragments from obese mice. Chemically defined, serum-free medium is used to ensure consistent process control, since it is well known that different lots of serum can significantly alter processes such as cell differentiation. Cell therapy bioprocesses that rely on serum often require expensive lot testing procedures upstream of the cell production process, increasing the complexity and cost of the overall process. Further, even though the patient will never be exposed to the factors used to treat the WAT ex vivo, a fully defined product will be helpful in ensuring patients and regulatory bodies that the product is defined, well characterized, and consistent lot-to-lot.

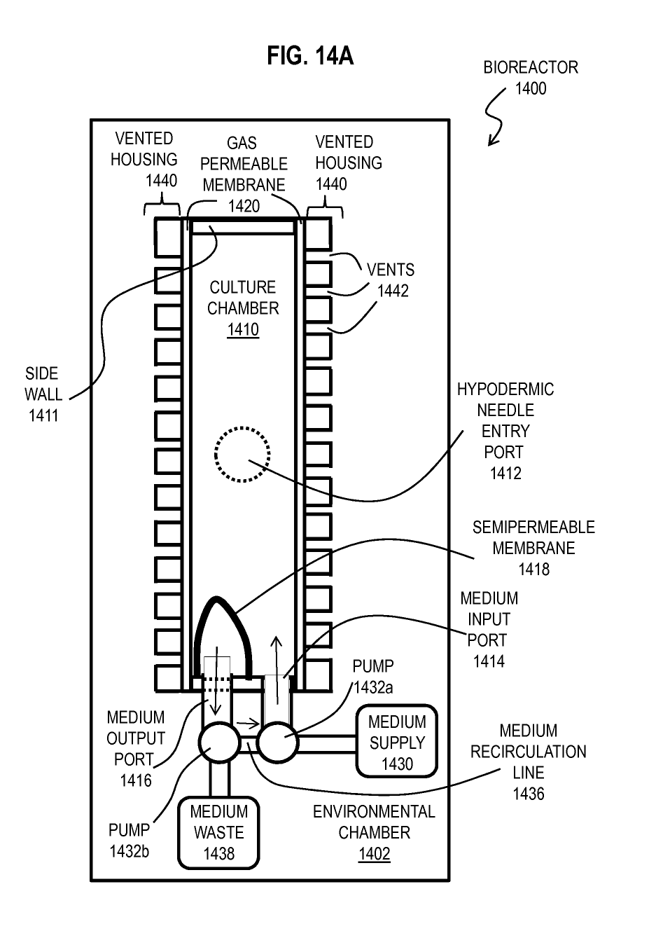

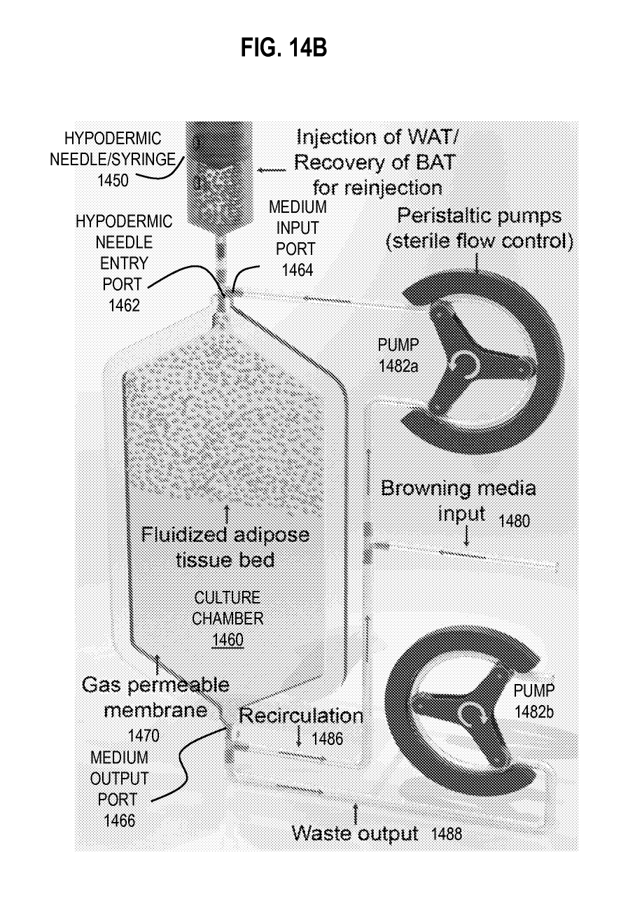

In certain embodiments, a method of treatment for a metabolic disorder (e.g., obesity, overweight, type 2 diabetes, metabolic syndrome, impaired glucose tolerance, insulin-resistance, dyslipidemia, cardiovascular disease, and hypertension) is provided by directly harvesting or isolating (e.g., by liposuction or surgical excision or other known methods in the art) white adipose tissue from a subject. This novel approach to increase BAT in humans occurs through ex vivo browning of adipose tissue, or “ThermoGraft” (FIG. 14). Subcutaneous WAT is first harvested from the patient utilizing techniques and tools of commonly performed autologous fat-transfer procedures. The WAT is aseptically transferred into a perfusion bioreactor that mimics native vascular and interstitial flow, and then exposed to culture conditions either in the presence of media comprising browning factors (e.g., norepinephrine or those listed in Table 1), or exposed to cold temperatures in a range from about 10° C. to about 40° C., about 15° C. to about 35° C., about 20° C. to about 35° C., about 25° C. to about 35° C., about 30° C. to about 35° C., about 15° C., about 20° C., about 25° C. to about 30° C., about 25° C., about 30° C., or about 35° C., or exposed to a combination of both browning factors and cold temperature to induce conversion of white adipose tissue fragments to brown adipose tissue fragments to induce development of UCP1-expressing brown adipocytes. Ranges for amount of browning factors are not limited to those listed in Table 1, are known in the art, and may vary according to factors such as the disease state, age, sex, and weight of the individual. The tissue is washed to remove the browning factor (e.g., norepinephrine or those listed in Table 1), withdrawn back into a fat transfer syringe, and then reimplanted back into subcutaneous WAT.

TABLE 1
List of factors to be investigated for optimizing ex vivo browning

| Tissue | Browning | Vascularization | Environment |
|---|---|---|---|
| maintenance: | factors: | factors: | factors: |
| Insulin* | Temperature | Vascular | Flow rate* |
| (0-1 μM) | (15-37 C.)* | endothelial | (1-100 mL/min) |
| Hydrocortisone* | Norepinephrine* | growth | Media exchange |
| (0-10 nM) | (0-10 μM) | factor (VEGF) | rate* |
| Sodium | Thyroxine (T3) | (0-50 nM) | (10-500%/day, |
| ascorbate* | (0-1 μM) | | i.e. up to |
| (0-25 μg/mL) | Retinoic Acid | Basic | 5 chamber |
| | (0-10 μM) | fibroblast | volume media |
| | Orexin | growth | changes daily) |
| | (0-10 μM) | factor (bFGF) ) | % CO₂* (0-10%) |
| | Rosiglitizone | (0-50 nM) | % O₂ (10-30%) |
| | (0-10 μM) | | |

*Primary factors of interest for minimum factor browing conditions

Basal Medium: Medium 100 (M199), or Dulbecco's modified eagle medium (DMEM) can serve as a basal medium to which browning and tissue maintenance factors are added. Other common mammalian cell culture media may be used, including but not limited to the following: BGJb (Fitton-Jackson Modification), BME, Brinster's BMOC-3, CMRL, CO2-Independent Medium, DMEM Media, DMEM/F-12 Media, F-10 Nutrient Mixture, F-12 Nutrient Mixture, Glasgow (G-MEM), Improved MEM, Iscove's (IMDM), Leibovitz's L-15, McCoy's 5A, MCDB 131, Media 199, Minimum Essential Media (MEM), Modified Eagle Medium (MEM), Opti-MEM® I, Other Basal Media, RPMI Medium 1640, Waymouth's MB 752/1, and Williams' Media E.

In certain embodiments, cell-specific media for the following cell types may be used:
Corneal Epithelial Cells
Fibroblasts
Hepatocytes
Keratinocytes
Mammary Epithelial Cells
Melanocytes
Microvascular Endothelial Cells
Large Vessel Endothelial Cells
Neuronal, Glial, and Neural Stem Cells
Skeletal Myoblasts
Smooth Muscle Cells

In certain embodiments, stem cell media formulations may include:
MesenPro RS™
StemPro® MSC SFM
StemPro® MSC SFM XenoFree
StemPro® BM Mesenchymal Stem Cells
StemPro®-34 SFM
Essential 8™ Medium

In certain embodiments, mixtures and variations of these are contemplated. It is also possible in certain embodiments to use patient blood or plasma as a “medium” and add factors to the blood (such as norepinephrine, etc.) or to mix in a portion of blood/plasma with the medium. One of ordinary skill in the art is not limited to the browning factors listed above in Table 1. Additional additives may include HEPES buffer and antibiotic or antibiotic/antimycotic solution (e.g., penicillin/streptomycin solution).

In these embodiments, a direct conversion of harvested WAT fragments to BAT occurs. Patients require two outpatient visits (one to obtain WAT tissue, one to inject the BAT grafts) involving subcutaneous needle procedures with local anesthesia. They need not undergo dramatic changes in food consumption habits or take daily pills. Moreover, the grafts are created from a patient's own tissue using endogenous stimuli, so nothing foreign is introduced to the body. BAT burns energy naturally in humans with no known side effects. Obese patients can readily have several liters of WAT harvested in a single harvest procedure, with easy recovery and minimal discomfort. Depending on those skilled in the art, this procedure may be modified to harvest less WAT or more if necessary. In certain embodiments, a typical fat harvest may be a 20-30 minute procedure (15 minutes for local tumescent anesthesia and 5-15 minutes for harvest), and the subsequent injection of ThermoGraft also takes around 30 minutes. Depending on those skilled in the art, this procedure may be modified to a shorter or longer time frame as required. The amount of BAT injected could be adjusted to control the rate of weight loss. One of ordinary skill in the art would adjust accordingly, taking into consideration the patient's general overall health and desired weight loss. Multiple injections could be performed if weight loss is insufficient or injected BAT loses thermogenic activity over time (fat transfer patients can have repeated procedures as desired). If weight loss is excessive (less likely since BAT is regulated by the body), BAT could be removed by fat harvesting.

In certain embodiments, the procedure adheres to the typical workflow for autologous fat transfer procedures. The surgeon transfers fat directly into a single-use browning bioreactor, using whatever fat harvesting device and method they prefer. The browning process can be fully automated, and when ready, the tissue can be directly harvested into fat transfer syringes for subcutaneous reimplantation. The compact cartridge design should allow for a bench top-sized incubator system that should easily fit in a doctor's office to accommodate numerous patient tissues, avoiding the need to transfer tissue to another location (such as a blood bank). Plastic surgeons or other fat-transfer specialists known in the art would purchase the devices and consumables and bill for procedures, similar to existing fat transfer devices and consumables. Plastic surgeons would be likely adopters of this method of treatment, as weight loss therapy represents a new high-value application of existing fat transfer skills. Collaboration between plastic surgeons and weight control specialists would help to optimize efficacy.

The disclosure provides for conditions in tissue, for example, mouse and human tissue, that are capable of converting whole fragments of subcutaneous adipose tissue to converted BAT whose UCP1 immunostaining, lipid droplet formation, and cellular remodeling characteristics are consistent with those exhibited by BAT. In an aspect, this conversion occurs in a single step. In another aspect, this conversion occurs in a single step without the need for isolation of individual stem cells and subsequent expansion which is low in yield and time consuming. In yet another aspect, this conversion occurs in a single step in a bioreactor without the need for isolation of individual stem cells and subsequent expansion which is low in yield and time consuming. In another aspect, methods described herein are capable of converting about 30% or more, about 40% or more, about 50% or more, about 60% or more, about 70% or more, about 80% or more, or about 90% or more of WAT to BAT or BAT-like tissue. In another aspect, methods described herein are capable of converting about 30% or more, about 40% or more, about 50% or more, about 60% or more, about 70% or more, about 80% or more, or about 90% or more of WAT to BAT or BAT-like tissue after about 1 week, about 2 weeks, about 3 weeks, about 4 weeks, about 5 weeks, about 6 weeks, about 7 weeks, about 8 weeks, about 12 weeks, about 6 months, or about 12 monthsfollowing autologous reimplantation. In another aspect, the converted BAT maintains its phenotype for at least about 1 week, at least about 2 weeks, at least about 3 weeks, at least about 4 weeks, at least about 5 weeks, at least about 6 weeks, at least about 7 weeks, at least about 8 weeks, at least about 10 weeks, at least about 6 months, or about 12 months or more following autologous reimplantation. In yet another aspect, the converted BAT maintains its phenotype for at least about 1 week, at least about 2 weeks, at least about 3 weeks, at least about 4 weeks, at least about 5 weeks, at least about 6 weeks, at least about 7 weeks, at least about 8 weeks, at least about 10 weeks, at least about 6 months, or at least about 12 months or more following autologous reimplantation as compared to the about one week shown using previous approaches for cell implantation as recognized by a person of skill in the art and as described herein. The exBAT approach provides a simple and sustained method for increasing BAT mass in vivo without exposure to systemic drugs.

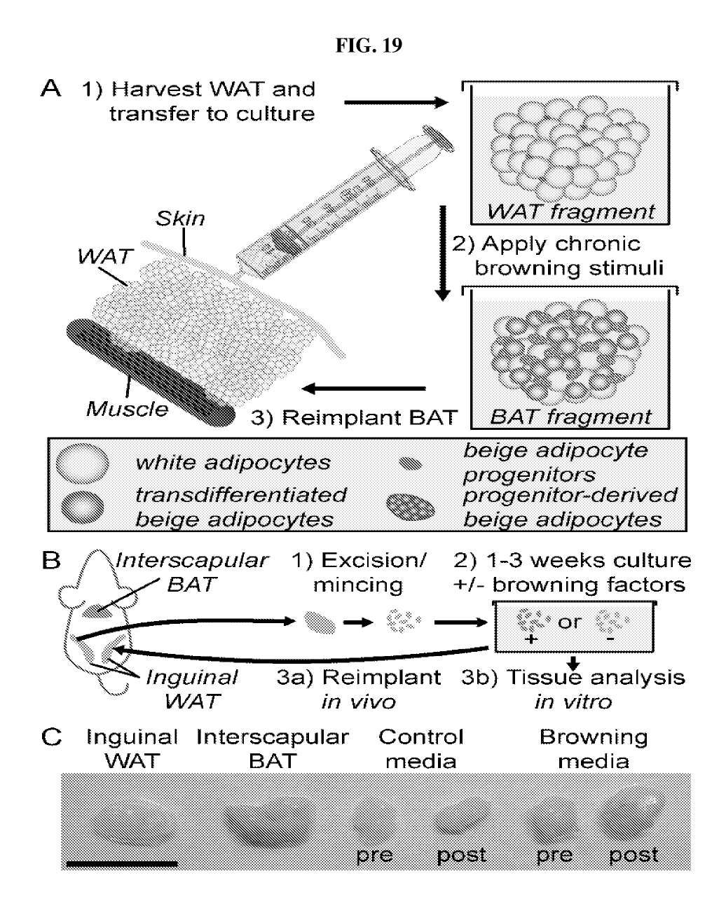

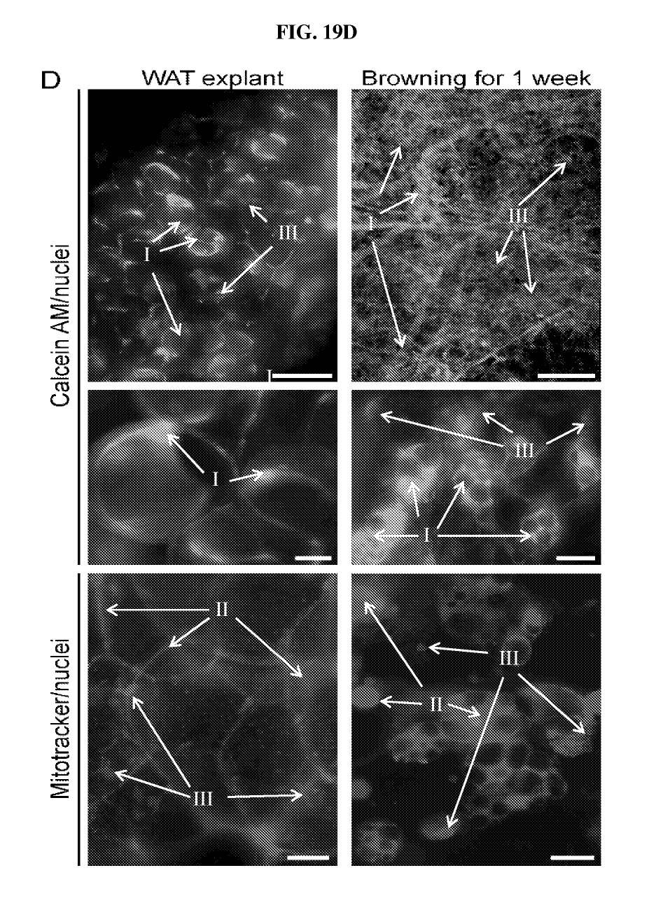

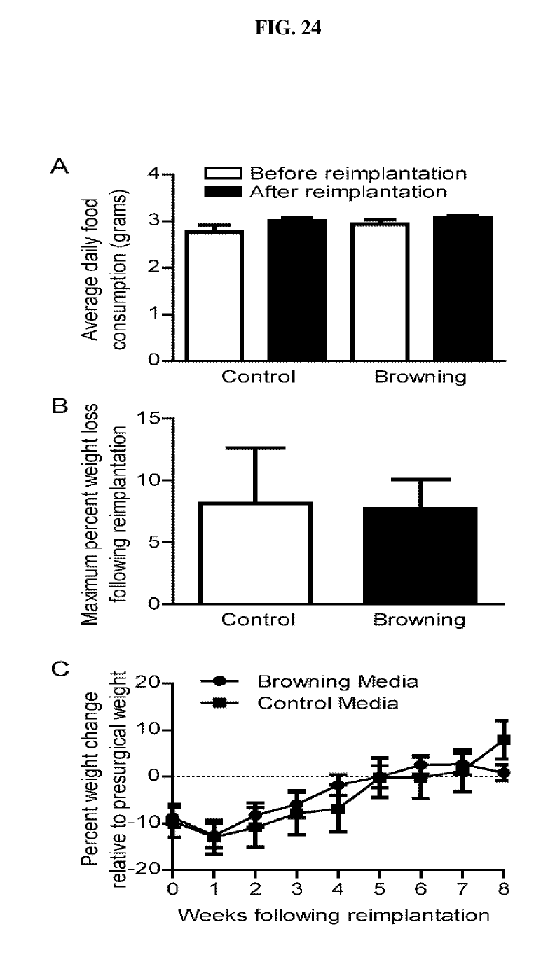

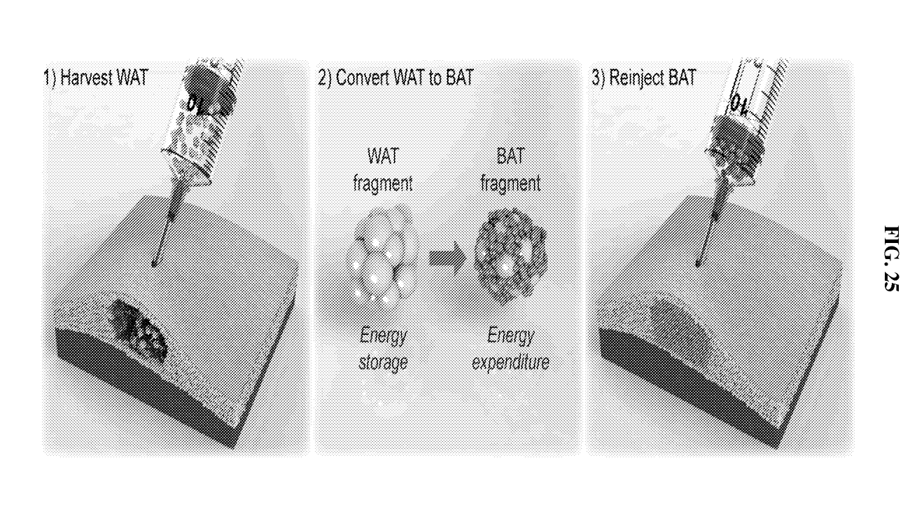

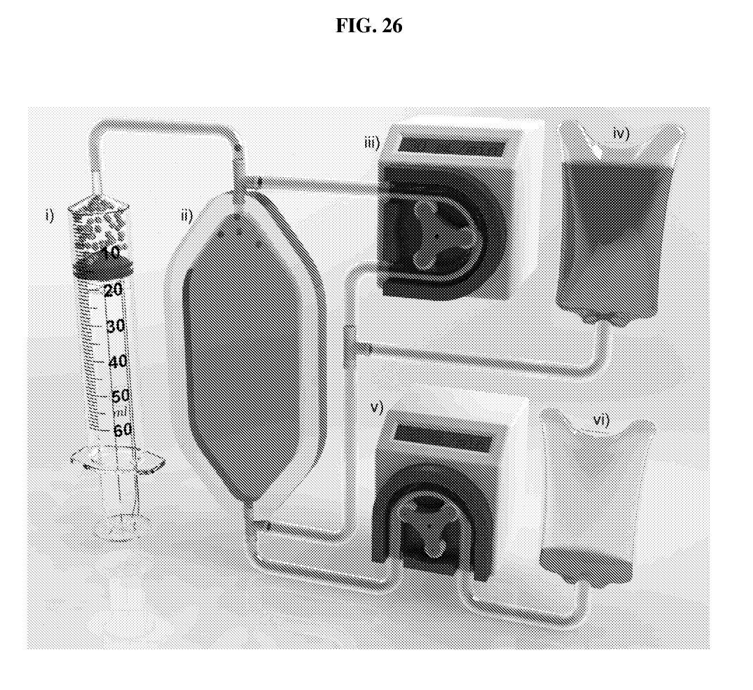

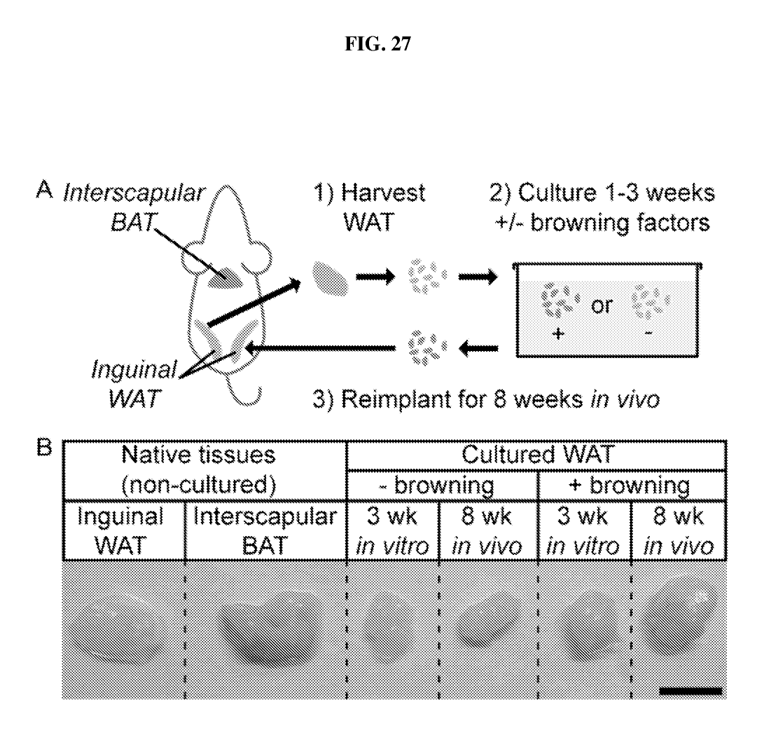

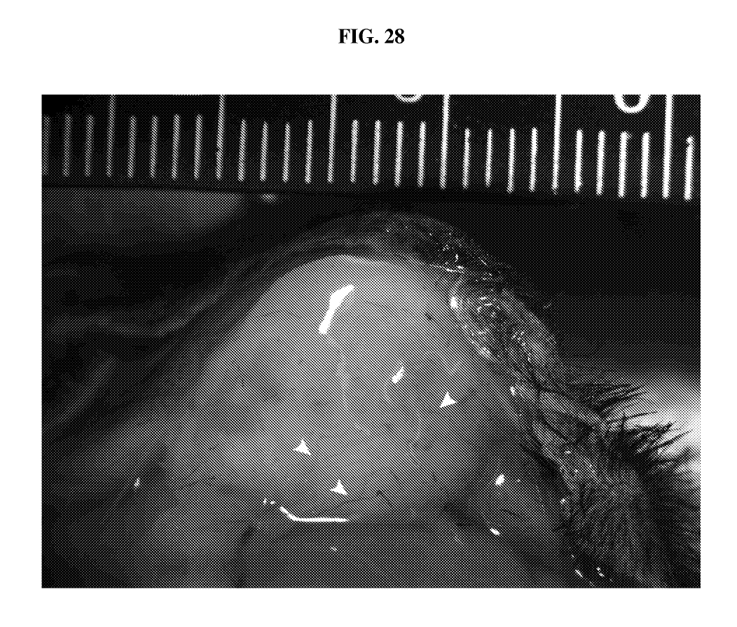

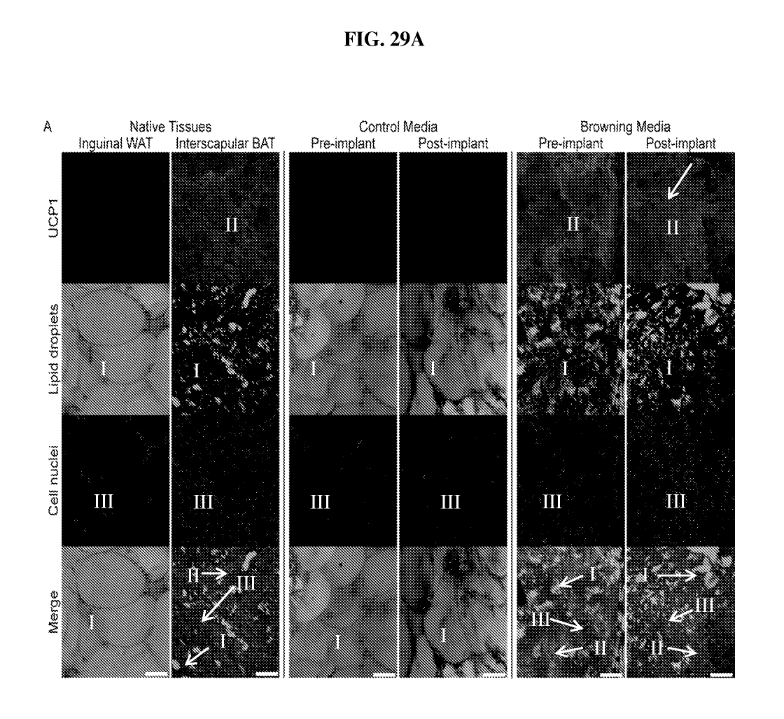

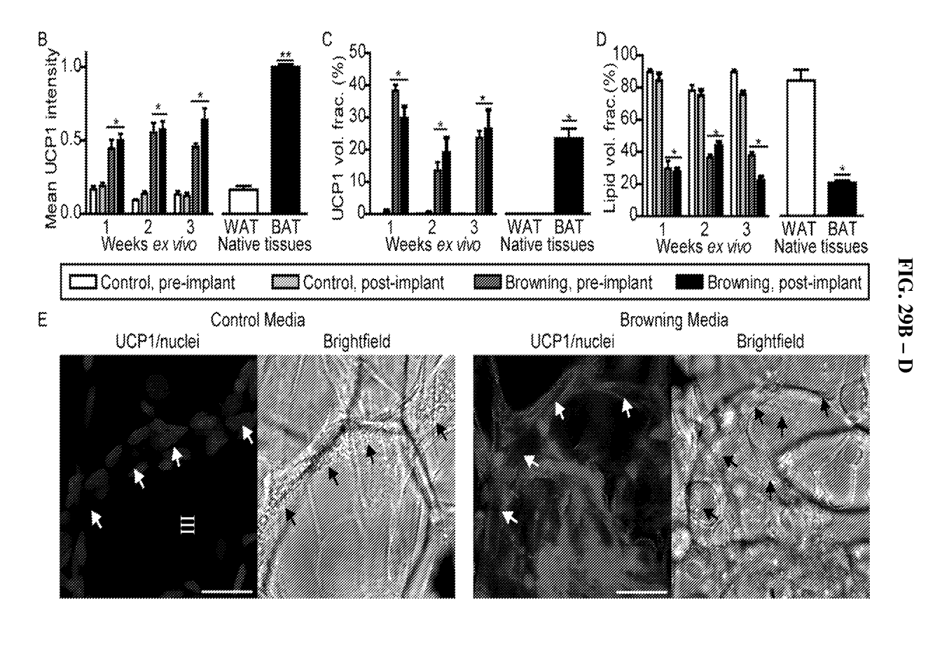

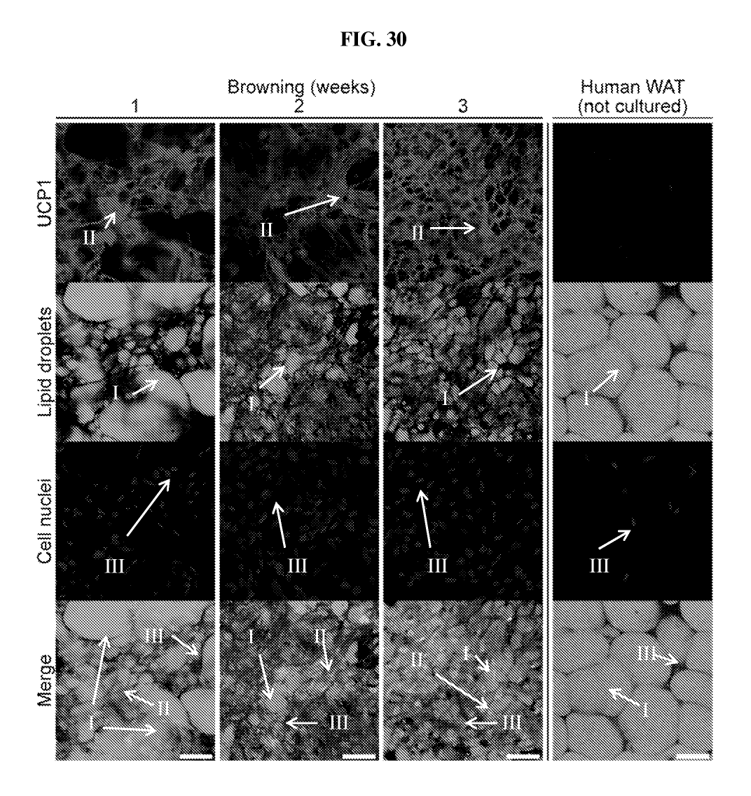

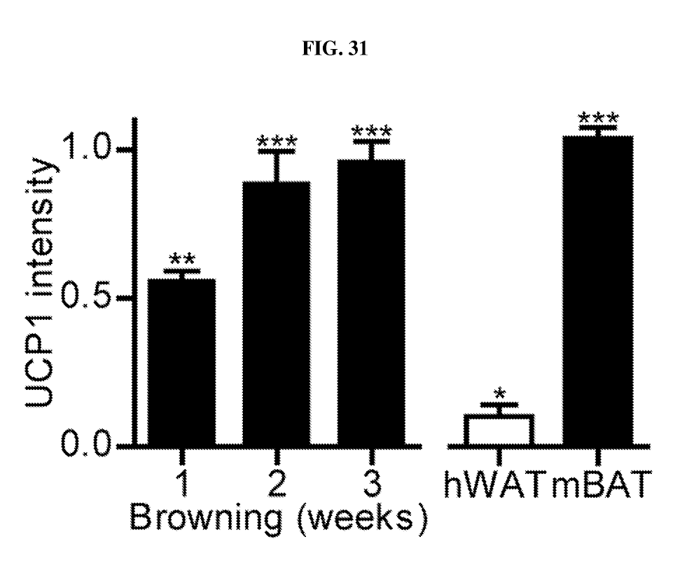

In certain embodiments, the disclosure provides for single step whole tissue browning. In an aspect, methods described herein include adding a single step of culture in browning media to the workflow for autologous fat grafting procedures. In an aspect, this allows for improved clinical implementation and production within the clinician's office. Unlike isolated cells in 2D culture, whole tissue fragments retain multiple cell types, 3D extracellular matrix scaffolding, and intact cellular niches. There are several different cell types and tissue structures involved in native browning (see, for example,; Qiu, Y., et al., Eosinophils and type 2 cytokine signaling in macrophages orchestrate development of functional beige fat. Cell, 2014. 157(6): p. 1292-308, which is herein incorporated by reference in its entirety) beyond adipocytes, it was observed that vessel-like structures appeared to remain viable during ex vivo culture (FIG. 19D), and that tissue fragments became revascularized after reimplantation (FIG. 21B). As described herein, the stability of BAT phenotype and high levels of UCP1 signal 8 weeks after reimplantation suggests that ex vivo stimulation with browning factors produced a more durable expression of UCP1 that more closely resembles permanent interscapular BAT than reversible beige fat. In an aspect, because UCP1 was highly expressed, the BAT-like tissue would be thermogenically competent. In FIG. 24, food consumption and body weight of the mice was measured weekly to determine if reimplanting browned WAT had any observable effect on weight loss or increased food consumption relative to tissues cultured in control media. All mice lost weight following the initial surgery to harvest inguinal WAT, which is a typical response to the stress of surgical procedures. Decreases in daily food consumption were not observed following initial surgery. At the time of reimplantation surgery, mice exhibited around 10% weight loss relative to their weight prior to WAT harvest (FIG. 24C). Mice in both browning and control conditions lost additional weight in the week following reimplantation, and slowly recovered to presurgical weight over the course of 8 weeks, with no statistical differences seen between groups. There were no statistically significant differences in daily food consumption or weight changes following reimplantation.

### 8. Implantation Procedures

Methods described herein can include implanting a population of microtissues such as BAMs or reimplantation of BAT fragments into a subject to be treated. The BAMs undergo adipogenesis prior to implantation. Once implanted, the BAMs undergo thermogenesis, increasing the metabolism of the subject. In addition to the treatment of metabolic syndrome, type 2 diabetes, obesity and insulin resistance in a subject, diseases associated with a lack of mitochondria, e.g., cancer, neurodegeneration, and aging can occur.

Methods for implanting BAMs and reimplantation of BAT fragments are known in the art, e.g., using a delivery system configured to allow the introduction of BAM and reimplantation of BAT fragments into a subject. BAT may be reimplanted back into subcutaneous WAT using a fat transfer syringe. Other delivery systems can include a reservoir containing a population of cells including BAMs, and a needle in fluid communication with the reservoir. Typically, the BAMs will be in a pharmaceutically acceptable carrier, with or without a scaffold, matrix, or other implantable device to which the cells can attach (examples include carriers made of collagen, fibronectin, elastin, cellulose acetate, cellulose nitrate, polysaccharide, fibrin, gelatin, and combinations thereof). Such delivery systems are also within the scope of the invention. Generally, such delivery systems are maintained in a sterile manner. Various routes of administration and various sites (e.g., renal subcapsular, subcutaneous, central nervous system [including intrathecal], intravascular, intrahepatic, intrasplanchnic, intraperitoneal [including intraomental], intramuscular implantation) can be used.

Generally, the cells will be implanted into the subject subcutaneously. In some embodiments, the BAMs that are implanted include at least 10⁶, 10⁷, 10⁸, 10⁹, or more cells. In other embodiments, the amount of BAT from 0.02-20 kilograms that is reimplanted back into the subcutaneous WAT of a patient could be adjusted to control the rate of weight loss. Multiple injections could be performed if weight loss is insufficient or injected BAT loses thermogenic activity over time (fat transfer patients can have repeated procedures as desired).

Where non-autologous, non-immunologically compatible cells including allogenic and xenogenic cells are used, an immunosuppressive compound, e.g., a drug or antibody, can be administered to the recipient subject at a dosage sufficient to reduce or inhibit rejection of the implanted microtissues. Dosage ranges for immunosuppressive drugs are known in the art. See, e.g., Freed et al., N. Engl. J. Med. 327:1549 (1992); Spencer et al., N. Engl. J. Med. 327:1541 (1992); Widner et al., N. Engl. J. Med. 327:1556 (1992). Dosage values may vary according to factors such as the disease state, age, sex, and weight of the individual.

In some embodiments, the methods include contacting, administering or expressing one or more other compounds in addition to the BAMs and BAT fragments, e.g., peroxisome proliferator-activated receptor gamma (PPARγ), Retinoid X receptor, alpha (RxRa), insulin, T3, a thiazolidinedione (TZD), retinoic acid, another BMP protein (e.g., BMP-1 or BMP-3), vitamin A, retinoic acid, insulin, glucocorticoid or agonist thereof, Wingless-type (Wnt), e.g., Wnt-1, Insulin-like Growth Factor-1 (IGF-1), or other growth factor, e.g., Epidermal growth factor (EGF), Fibroblast growth factor (FGF), Transforming growth factor (TGF)-α, TGF-β, Tumor necrosis factor alpha (TNFα), Macrophage colony stimulating factor (MCSF), Vascular endothelial growth factor (VEGF) and/or Platelet-derived growth factor (PDGF). In other embodiments, the methods include administering the compound in combination with a second treatment, e.g., a second treatment for obesity or a related disorder such as diabetes. For example, the second treatment can be insulin, orlistat, phendimetrazine, and/or phentermine.

Finally, in yet other embodiments devices for the collection and packing together of microtis sues from solution and devices for generation of BAT fragments allow for direct injection into the subject.

### 9. Assessment/Validation of Treatment

In some embodiments, the methods described can include assessing the amount or activity of BAT in the subject before and after treatment with the microtissues and recording these observations. In some embodiments, BAMs are administered to the subject and an effective implantation of BAM will result in increased BAT levels and/or activity. In some embodiments, the subject will show reduced symptoms.

These assessments can be used to determine the future course of treatment for the subject. For example, assessments of BAT activity can be made at various time points after treatment to help determine how the patient is responding and whether a second treatment of administering BAM is advisable, for example if BAT activity begins to fall to pretreatment levels, or if symptoms reoccur. Based on the results of the assessment, treatment may be continued without change, continued with change (e.g., additional treatment or more aggressive treatment), or treatment can be stopped. In some embodiments, the methods include one or more additional rounds of implantation of BAMs, e.g., to increase brown adipose levels, thermogenesis and metabolism, e.g., to maintain or further reduce obesity in the subject.

In some embodiments, assessment can include determining the subject's weight or BMI before and/or after treatment, and comparing the subject's weight or BMI before treatment to the weight or BMI after treatment. An indication of success would be observation of a decrease in weight or BMI. In some embodiments, the treatment is administered one or more additional times until a target weight or BMI is achieved. Alternatively, measurements of girth can be used, e.g., waist, chest, hip, thigh, or arm circumference.

### 10. Administration

Introduction of the microtissue, e.g., BAMs or BAT fragments into a subject can be carried out by direct surgical implantation or by introduction with the assistance of a surgical aid such as a catheter-based delivery system or injection by needle. In some embodiments, the cells carried by the substrate are not encapsulated or surface coated (as is done with other types of artificial organs) so that, once implanted, the stem cells are in direct contact with the host (host tissue, host blood, etc.).

For example, a microtissue, e.g., BAMs or BAT fragments of some of one of the embodiments may be implanted in a muscle such as an abdominal or lumbar muscle, or even an extremity muscle such as a quadricep or hamstring muscle. Muscle is a useful implantation region because it is highly vascularized. For muscle implantation, a small incision may be made through the muscle fascia so that the substrate may be implanted directly into the muscle tissue itself to maximize potential vascular contact. In other embodiments, the microtissue is implanted in a fatty layer below the skin. In yet other embodiments, the BAT fragments are reimplanted back into subcutaneous WAT.

### 11. Effective Dose

Toxicity and therapeutic efficacy of the pharmaceutical compositions of microtissues or BAT fragments described herein can be determined by standard pharmaceutical procedures, using either cells in culture or experimental animals to determine the LD₅₀ (the dose lethal to 50% of the population) and the ED₅₀ (the dose therapeutically effective in 50% of the population). The dose ratio between toxic and therapeutic effects is the therapeutic index and can be expressed as the ratio LD₅₀/ED₅₀.

Data obtained from cell culture assays and further animal studies can be used in formulating a range of dosage for use in humans. The dosage may vary within this range depending upon the dosage form employed and the route of administration utilized. For any pharmaceutical composition of microtissues or BAT fragments used in the methods described herein, the therapeutically effective dose can be estimated initially from cell culture assays. Such information can be used to more accurately determine useful doses in humans.

The formulations comprising the microtissues or BAT fragments and routes of administration can be tailored to the disease or disorder being treated, and for the specific human being treated. A subject can receive a dose of the formulation comprising the microtissues once or twice or more daily for one week, one month, six months, one year, or more. The treatment can continue indefinitely, such as throughout the lifetime of the human. Treatment can be administered at regular or irregular intervals (once every other day or twice per week), and the dosage and timing of the administration can be adjusted throughout the course of the treatment. The dosage can remain constant over the course of the treatment regimen, or it can be decreased or increased over the course of the treatment. In some embodiments, the formulation comprising the microtissues can comprise other drugs known to treat the targeted metabolic disease or disorder. Up to 20 kg fat tissue could be harvested in a single procedure (fragment sizes are 2-6 mm in diameter). The harvested fat could be cryopreserved for later injections either before or after browning in the bioreactor. Injections could be performed at various frequencies, probably a minimum interval of once daily. Longer intervals would be preferred (weekly, monthly, every 6 months etc.) depending on efficacy.

Generally the dosage facilitates an intended purpose for both prophylaxis and treatment without undesirable side effects, such as toxicity, irritation or allergic response. Although individual needs may vary, the determination of optimal ranges for effective amounts of formulations is within the skill of the art. Human doses can readily be extrapolated from animal studies (Katocs et al., Chapter 27 in: Remington's Pharmaceutical Sciences, 18th ed., Gennaro, ed., Mack Publishing Co., Easton, Pa., 1990). Generally, the dosage required to provide an effective amount of a formulation, which can be adjusted by one skilled in the art, will vary depending on several factors, including the age, health, physical condition, weight, type and extent of the disease or disorder of the recipient, frequency of treatment, the nature of concurrent therapy, if required, and the nature and scope of the desired effect(s) (Nies et al., Chapter 3, in: Goodman & Gilman's “The Pharmacological Basis of Therapeutics,” 9th ed., Hardman et al., eds., McGraw-Hill, New York, N.Y., 1996).

### 12. Bioreactor Systems

FIG. 14A is a block diagram that illustrates an example bioreactor system 1400 for browning adipose tissue, according to an embodiment. In other embodiments, the bioreactor1400 or portions thereof are used to culture other tissues. FIG. 14A indicates a plan view of a cross section of the device looking down from above.

At the core of the system is a culture chamber 1410 enclosed, at least in part by one or more gas-permeable membranes 1420. Any suitable material for the reaction or cultures in the chamber may be used, as described in more detail below. In the illustrated device, the culture chamber includes side walls 1411, at top and bottom of drawing of culture chamber 1410. The side walls 1411 are made of a rigid material such as plastic that is inert with respect to any tissue to be cultured in the culture chamber. In other embodiments, a bag of the gas-permeable membrane is used and the side walls 1411 of rigid material are omitted. In some embodiments, the shape of the culture chamber is controlled by an external rigid housing 1440 that prevents the gas-permeable membranes 1420, which are often elastic and fragile, from deforming. When present, the housing 1440 includes vents 1442 configured to allow gas to reach the surface of the gas-permeable membrane, at least in some locations. The housing can be an integral unit, in some embodiments, by connecting in other planes from the cross section shown in FIG. 14A.

The culture chamber 1410 includes an input port 1414 to allow the passage of a culturing medium into the chamber 1410; and an output port 1416 to allow the passage of depleted or waste-laden medium out of the chamber 1410. The chamber 1410 can be formed as a single channel (as shown) or multiple channels, all in fluid communication with both the input port 1414 and output port 1416.

Tissue to be cultured (e.g., white adipose tissue, WAT) is introduced into the chamber 1410 through an access port, such as the hypodermic needle entry port 1412 along a top surface of the chamber, shown as a dotted circle because the port is not necessarily in the cross section depicted. Tissue that is a product of the culturing (e.g., brown adipose tissue, BAT) is removed from the chamber through the same or similar port. For example, a hypodermic needle is connected to a syringe that is full of WAT. The needle is inserted into the port 1412 and the syringe plunger pushed to inject WAT into the chamber 1410. After sufficient incubation to form brown adipose tissues (BAT), the needle is introduced, with the plunger of the syringe down; the plunger is pulled, and BAT is extracted from the chamber into the syringe through the needle in port 1412. Tissue passed through a hypodermic needle is fragmented, depending on the tissue pliability. For adipose tissue, tissue fragments of up to 6 millimeters can pass through some needles into the chamber 1410.

The output port 1416 is separated from the tissue access port (e.g., port 1412) so that tissue does not pass through the output port 1416. When used with tissue that floats or sinks in the medium, tissue loss can be inhibited or eliminated by having the output port 1416 vertically separated from the tissue access port 1412 (e.g., below the access port 1412 for floating tissue fragments, like adipose tissue fragments, or above the access port 1412 for sinking tissue, such as heart muscle tissue). In some embodiments, the access port 1412 is separated from the output port 1416 by a semi-permeable membrane 1418 that allows a carrier fluid and at least some waste materials to pass but does not allow tissue fragments to pass. The vertical separation is indicated in FIG. 14A, by the dotted line blocking the output port 1416 to indicate the output port 1416 is not necessarily in the plane of the cross section with the input port 1414.

The system 1400 includes a medium supply 1430, such as a fluid supply bag; and medium waste receptacle 438 wherein is deposited the medium after interaction with the cultured tissue fragments. The fluid in the supply bag typically includes nutrients such as glucose to feed the tissue fragments, and any other culturing factors (such as one or more adipose browning factors). The medium flowing through the output port 1416 is at least partially depleted in nutrients and culturing factors and at least partially laden with waste products form the tissue fragments. In some embodiments, only a portion of the fluid passing through the output port 1416 is deposited in a waste receptacle 1438, and the rest is recirculated through line 1436 the input port 1414. The portion recycled depends on the rate of nutrient and factor depletion and the rate of waste production and the rate of introduction of new medium from the supply 1430. In some embodiments the system 1400 includes one or more pumps (e.g., pumps 1432a and 1432b) to control the rate of supply and removal of the medium, either together or individually.

The system 1400 includes an environmental chamber 1402 in which the culture chamber 1410 is disposed. The environmental chamber includes controls for the gas mixture or temperature or both for the culturing of the tissue. For example, the gas mixture includes a supply of oxygen and a means to remove the carbon dioxide.

FIG. 14B is a photograph of bioreactor device for ex vivo browning of WAT fragments to convert WAT to BAT in the presence of browning media, according to an embodiment. This embodiment includes a bag of gas-permeable membrane 1470 enclosing a culture chamber 1460, and a hypodermic needle and syringe 1450 inserted into access port 1462. The depicted device includes a medium supply 1480, input port 1464, output port 1466, recirculation line 1486, and peristaltic pumps 1482a and 1482b. This embodiment cultures adipose tissue which floats in the aqueous medium. Thus an output port 1466 at the bottom of the chamber 1460 will be separated from the tissue fragments. As a consequence, a semipermeable membrane 1418 is not advantageous; and, thus, is omitted.

In certain embodiments, a prototype single use “cartridge” integrating a gas-permeable perfusion culture chamber, prefilled media and wash reagent bags, and a waste reservoir bag is provided. Gas permeable membranes may include silicone rubber (e.g., polydimethylsiloxane), fluorinated ethylene propylene (FEP), or polyolefin. Optional membranes permeable to water/solutes include cellulose, polysulfone, polyacrylonitrile or polyamide. The culture chambers are maintained in a temperature- and gas-controlled incubator, while media bags are stored in a separate refrigerated compartment to preserve factors such as NE which can degrade in warm media. A peristaltic pump delivers media in a closed, sterile manner to the culture chamber, which is stored vertically to create an “inverted fluidized bed” to perfuse the adipose explants and mimic vascular flow (i.e., the adipose fragments float to the top of the chamber while medium flows downwards through the bed of tissue). The fluid circuit is designed to allow for control over the percentage of recirculation versus fresh medium that is flowed into the chamber, in order to maintain stable culture conditions over time (i.e., there is a continuous fractional media change throughout culture). Media recirculation is utilized as autocrine and paracrine factors secreted by the tissue are involved in tissue level changes in native browning (for example, VEGF is secreted under adrenergic stimuli to induce angiogenesis surrounding developing BAT cells).

To support oxygenation of the tissue, the chamber is enclosed on both sides by a thin layer of biocompatible and highly gas-permeable FDA approved silicone elastomer. Since the thin silicone membrane is highly elastic and fragile, the chamber is enclosed within a plastic housing with gratings (vents) that expose the membrane surface while maintaining the chamber geometry such that a constant distance for oxygen and CO₂ diffusion is maintained across the entire chamber volume. The silicone elastomer is autoclavable and the housing can consist of a variety of autoclavable plastics (such as Ultem, PEEK, or polycarbonate). Current prototypes have been fabricated by CNC milling and 3D printing. The simple design could be readily made by injection molding. The two halves of the housing can be ultrasonically welded to securely encase the silicone chamber. A humid environment is not required as the silicone membrane has very low water permeability. In the future, gas lines could be integrated into the cartridge to improve efficiency.

The amount of WAT tissue that can be added to a given chamber volume was designed to correspond to the native ratio of adipose tissue mass to total extracellular water (˜0.5 g WAT per mL media). Thus for chamber volume of approximately 90 mL (with a device footprint of approximately 4″×8″×0.15″), ˜29 g of WAT can be added, with ˜58 mL of media circulating within the chamber. The chamber area can be enlarged or several chambers arrayed to accommodate larger volumes of tissue. To determine optimal flow rates and inlet/outlet designs to evenly perfuse the chamber, we have performed initial design studies using solid works flow simulation to model both fluid flow and fat particle motion incorporating gravity forces.

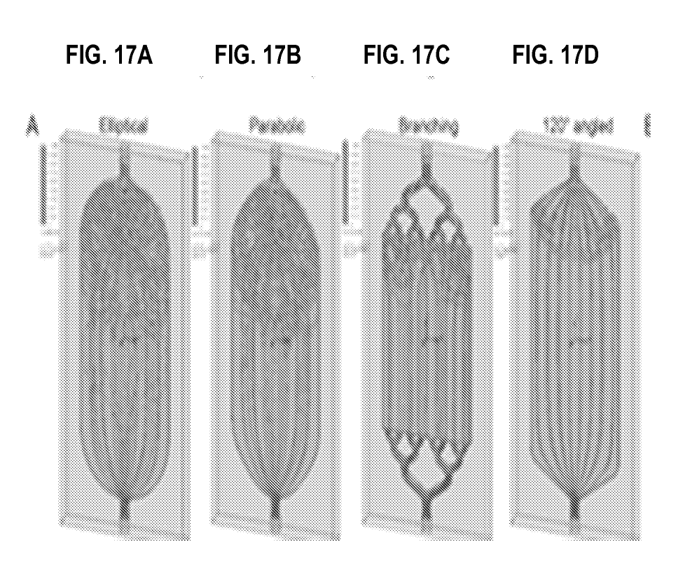

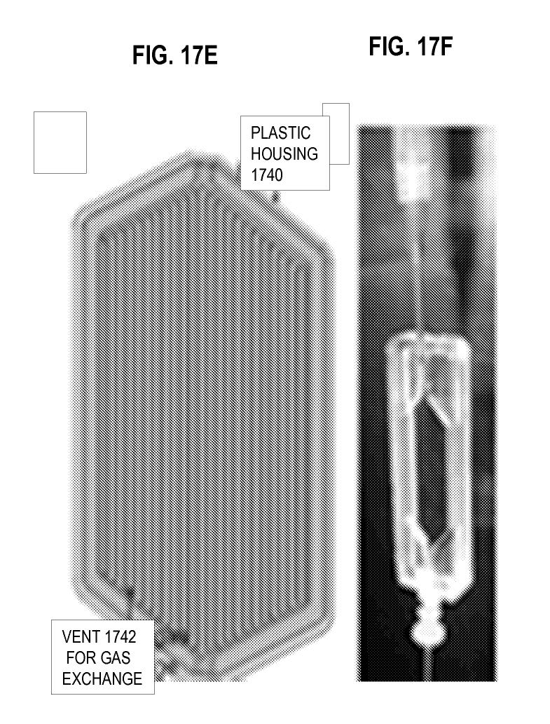

FIG. 17A through FIG. 17F are diagrams that illustrate example variations in devices for automated point-of-care culturing, according to various embodiments. FIG. 17A through FIG. 17D depict example embodiments that include a culture chamber made up of multiple channels, in four different configurations. Modeling was performed to determine whether any advantage is obtained in flow past tissue fragments and commensurate nutrient and factor supply, waste removal, and separation of tissue from the output port. FIG. 17E is a block diagram that illustrates an example plastic housing 1740 with vents 1742 for gas exchange that can be used with any of the embodiments of FIG. 17A through FIG. 17D. FIG. 17F is a photograph that illustrates an example single-use, transparent, plastic cartridge with access port, input port and output port.

### 13. Pharmaceutical Compositions

Pharmaceutical compositions for use in the present methods include therapeutically effective amounts of any type of microtissues, e.g., BAMs (therapeutic agent) or BAT fragments in an amount sufficient to prevent or treat the diseases described herein in a subject, formulated for local or systemic administration. The subject is preferably a human but can be non-human as well. A suitable subject can be an individual who is suspected of having, has been diagnosed as having, or is at risk of developing one of the described diseases, obesity or type 2 diabetes.

The therapeutic agents can also be mixed with diluents or excipients which are compatible and physiologically tolerable as selected in accordance with the route of administration and standard pharmaceutical practice. Suitable diluents and excipients are, for example, water, saline, dextrose, glycerol, or the like, and combinations thereof. In addition, if desired, the compositions may contain minor amounts of auxiliary substances such as wetting or emulsifying agents, stabilizing or pH buffering agents.

The therapeutic agents of the present invention may be administered by any suitable means. For in vivo administration, the pharmaceutical compositions are preferably administered parenterally, i.e., intraarticularly, intravenously, intraperitoneally, subcutaneously, or intramuscularly. In particular embodiments, the pharmaceutical compositions are administered intravenously or intraperitoneally by a bolus injection. (Stadler et al., U.S. Pat. No. 5,286,634.) For the prevention or treatment of disease, the appropriate dosage will depend on the severity of the disease, whether the therapeutic agent is administered for protective or therapeutic purposes, previous therapy, the patient's clinical history and response to the therapeutic agent, and the discretion of the attending physician.

### 14. Kits

The present invention may include kits. In some embodiments, the kits can include (1) pharmaceutical compositions comprising the microtissues, e.g., BAMs or BAT fragments; (2) a device for administering the pharmaceutical composition comprising the microtissues, e.g., BAMs or BAT fragments to a subject; (4) instructions for administration; and optionally (5) one or more differentiation induction cocktails or browning media.

In some embodiments, the kits can include (1) pharmaceutical compositions comprising the microtissues, e.g., BAMs or BAT fragments; (2) a device for administering the pharmaceutical compositions comprising the microtissues, e.g., BAMs or BAT fragments to a subject; and (3) instructions for administration. Embodiments in which two or more, including all, of the components are found in the same container are included.

When a kit is supplied, it may further contain other therapeutic agents for treating the targeted metabolic disease other than the microtissues, e.g., BAMs or BAT fragments. The different components of the pharmaceutical compositions included can be packaged in separate containers and admixed immediately before use. Such packaging of the components separately can permit long term storage without losing the active components' functions. When more than one therapeutic agent is included in addition to microtissues or BAT fragments, in a particular kit, they may be (1) packaged separately and admixed separately with appropriate (similar of different, but compatible) adjuvants or excipients immediately before use, (2) packaged together and admixed together immediately before use, or (3) packaged separately and admixed together immediately before use. If the chosen compounds will remain stable after admixing, the compounds may be admixed at a time before use other than immediately before use, including, for example, minutes, hours, days, months, years, and at the time of manufacture.

The compositions included in particular kits of the present invention can be supplied in containers of any sort such that the life of the different components are optimally preserved and are not adsorbed or altered by the materials of the container. Suitable materials for these containers may include, for example, glass, organic polymers (e.g., polycarbonate and polystyrene), ceramic, metal (e.g., aluminum), an alloy, or any other material typically employed to hold similar reagents. Exemplary containers may include, without limitation, test tubes, vials, flasks, bottles, syringes, and the like.

As stated above, the kits can also be supplied with instructional materials. These instructions may be printed and/or may be supplied, without limitation, as an electronic-readable medium, such as a floppy disc, a CD-ROM, a DVD, a Zip disc, a video cassette, an audiotape, and a flash memory device. Alternatively, instructions may be published on an Internet web site or may be distributed to the user as an electronic mail.

The kits also include kits for the treatment or prevention of metabolic disorders such type 2 diabetes and obesity.

### EXAMPLES

The invention is illustrated herein by the experiments described by the following examples, which should not be construed as limiting. The contents of all references, pending patent applications and published patents, cited throughout this application are hereby expressly incorporated by reference. Those skilled in the art will understand that this invention may be embodied in many different forms and should not be construed as limited to the embodiments set forth herein. Rather, these embodiments are provided so that this disclosure will fully convey the invention to those skilled in the art. Many modifications and other embodiments of the invention will come to mind in one skilled in the art to which this invention pertains having the benefit of the teachings presented in the foregoing description. Although specific terms are employed, they are used as in the art unless otherwise indicated.

### Isolated Stem Cell-Based Approach

### Example 1

### Materials and Methods

Chemical Reagents—All chemical reagents were obtained from Sigma Aldrich.

Animals—All procedures involving animals were approved by the Institutional Animal Care and Use Committee at Columbia University. Mice were maintained under appropriate barrier conditions in a 12 hr light-dark cycle and received food and water ad libitum. Mice, in particular the C57BL/6 strain, were used. When fed a high-fat diet, C57B1/6 mice became obese and developed symptoms of type 2 diabetes including reduced glucose tolerance and insulin sensitivity. This is referred to as the diet-induced obesity (DIO) model, and several companies (e.g., Jackson, Taconic) sell DIO C57BL/6 mice at varying ages and time on a high-fat diet specifically for research into obesity and type 2 diabetes. The DIO C57BL/6 mouse was used.

Each mouse was subjected to two surgical procedures: one procedure to obtain a small amount (˜100 mg) of inguinal sWAT from which stem cells were obtained to be used to create the graft, and a second procedure to reimplant the graft in the inguinal sWAT depots. There will be approximately 1 month between procedures to allow for the expansion of the mouse stem cells and fabrication/differentiation of the grafts. The animals were given approximately 2 weeks following arrival in the barrier to acclimate before the first surgery to extract sWAT for obtaining stem cells. Prior to the first surgical procedure mice were weighed and a baseline glucose tolerance test (GTT), insulin sensitivity test (ITT), and lipid panel (cholesterol, nonesterified free fatty acids (NEFA), and triglicerides) were performed.

In the first surgical procedure a small amount (˜100 mg) of subcutaneous white adipose tissue (sWAT) was extracted from the inguinal depot (located above the hindquarters) to obtain stem cells or tissue from which the engineered BAT grafts were constructed. A 1 cm-long linear cut was made along midsagittal line of dorsum above the hindquarters, exposing the two inguinal sWAT depots, one on each side of the incision. A small portion of the inguinal sWAT was excised from the depot on one side of the animal using a fine point #11 scalpel blade to cut the fascia, and then the sWAT was removed using tissue gripping forceps, detaching it from the surrounding tissue using the scalpel blade. The extracted sWAT was aseptically transferred to a sterile 1.5 mL centrifuge for processing to obtain stem cells. The incision was then closed using 5 mm autoclips.

In the second procedure (˜1 month following the first procedure), the engineered BAT grafts were injected back into the inguinal depots, delivering approximately 10-200 microliters of grafts into each depot. A 1 cm-long linear cut was made along midsagittal line of dorsum above the hindquarters at the site of the first incision, exposing the two inguinal sWAT depots, one on each side of the incision. A tiny hole was created in the fascia surrounding the inguinal depot using a sterile 18-gauge needle, and then a sterile pipette tip was used to inject the engineered grafts through the hole and into the inguinal sWAT. Approximately 10-200 microliters of graft was injected into each of the two depots. The incision was closed using 5 mm autoclips.

Cells—Human ASC (obtained from Promocell) and mouse ASC (isolated from C57/BL6 mice) were cultured in mesenchymal stem cell growth media (Promocell) in culture flasks; media was changed twice weekly and the cells routinely passaged at 70-90% confluence. Human umbilical vein endothelial cells (obtained from Promocell) and mouse endothelial cells (isolated from mouse tissue using magnetic bead sorting) were cultured in endothelial cell growth medium/medium 2 (Promocell) in culture flasks; media was changed 2-3 times weekly and the cells routinely passaged at 70-90%. Mouse EC were isolated using magnetic beads. Beads were pre-coated with antibody by mixing 1˜3 ug mouse anti-PECAM-1 monoclonal antibody in sterile PBS per 25 ul of pre-washed and resuspended Dynabeads (CELLectin™ Biotin Binder Dynabeads, Invitrogen Dynal AS, Oslo, Norway), then incubating on a rotation mixer for at least 2 hours at room temperature. Free antibody was removed by washing twice for 5 min. Cell samples were mixed with pre-coated beads thoroughly and incubated for 2 hours at 2° C. to 8° C. on a rotation mixer. ECs were selected using the magnet (Invitrogen) for 2 min. The magnetically separated materials were washed three times in 0.1% BSA in PBS, pH 7.4 and plated in cell culture flasks in EC culture medium.

Immunofluorescence—Immunostaining was performed using standard techniques. Cells were first fixed using 4% paraformaldehyde overnight and permeabilized for 5 min using triton-x 100. Primary antibodies against UCP1 (#ab10983 Rabbit polyclonal to UCP1, ABCAM) and beta 3 adrenergic receptor (A4854-50UL, rabbit polyclonal to beta 3 Adrenergic Receptor antibody, Sigma) were incubated for 2 hours at room temperature or overnight at 4C. A fluorescent secondary antibody (Alexa Fluor® 555 labeled goat anti-rabbit, Life Technologies) was then incubated at room temperature for 30-60 minutes. The cells were then images on a Leica DMI 6000b inverted fluorescence microscope with a rhodamine filter (N3, filter cube, Leica) using uniform illumination and exposure settings for all samples. Samples processed without primary and without primary and secondary antibodies were prepared as controls and imaged using the same settings.

### Example 2

### Production of iBAMS Using Isolation and Expansion of ASCs and ECs and Formation and Differentiation of 3D Cell Aggregates

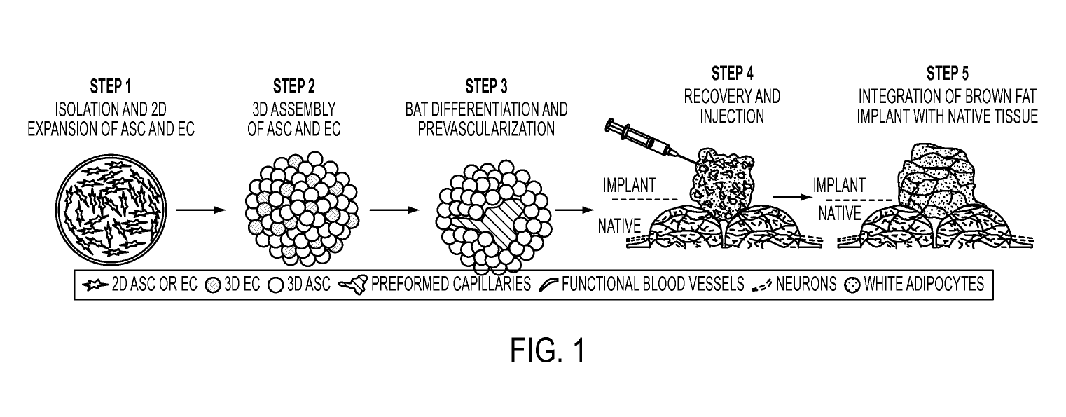

In some embodiments, the BAMs were produced by the following process as shown in FIG. 1.

Step 1: Isolation of stem cells. A patient fat biopsy was obtained by liposuction or surgical excision and collagenase or Liberase (Roche) at 10-100 Wuensch Units/mL used to digest the connective tissue. The tissue digest was then filtered and centrifuged to obtain the stromal vascular fraction (SVC), which consists of the ASC and EC. The ASC and EC were separated using antibodies against an EC-specific marker (e.g., CD31 protein) coupled to magnetic beads for magnetic sorting or fluorophores for fluorescence activated cells sorting (FACS). FIG. 1A.

Step 2: Expansion of ASC and EC. ASC and EC were expanded on traditional 2D culture surfaces using growth media (mesenchymal stem cell (MSC) growth media kit, endothelial cell growth medium 2 kit, Promocell) optimized for proliferating the cells while maintaining the ability for ASC to differentiate into BAT-like cells and ECs to form blood vessel structures. FIG. 1B.

Step 3: Formation and Development of BAMs in 3D culture. ASC and ECs were removed from the 2D culture surface and mixed together in a given ratio (such as 1:3 EC:ASC) and number of cells per volume of solution (e.g. 20 microliters for an array that fits in 24-well tissue culture plates), such that the total number of cells added to the array equals the number desired per aggregate times the number of microwells in the array (e.g. 200,000 cells in 20 microliters on an array with 1000 microwells to obtain 200 cells/aggregate). The cell suspension was then placed on an array of alginate hydrogel-based microwells, such that a specific number of cells (i.e. 200-5000 cells) fell onto each well by gravity. The non-adhesive hydrogel surface allows the cells to form a 3D aggregate in each microwell. The culture media was supplemented with a set of differentiation factors selected from a group including but not limited to the following drugs and growth factors: Dexamethasone, Indomethacin, Insulin, Isobutylmethylxanthine (IBMX), Rosiglitazone, Sodium Ascorbate, Triiodothyronine (T3), CL316,243, orexin, irisin, bone morphogenetic protein 7 (BMP7), fibroblast growth factor 21 (FGF21), vascular endothelial growth factor (VEGF), basic fibroblast growth factor (bFGF), and phorbol myristate acetate (PMA). During the aggregation process, ECs migrated to the middle of the BAM with ASC remaining on the outside. The 3D conformation of the aggregates along with factors in the media promoted collagen production, close cell-cell association, and rounded cell shape, which in turn promoted vascularization of EC and differentiation of the ASC to brown fat. The vascularization factors present in the media induced the EC to form open capillary structures with a fluid filled lumen. The brown adipogenic factors in the media promoted production of thermogenic machinery (for example increased numbers of mitochondria and UCP1 levels) and brown fat specific markers. The differentiation process can be carried out from several days up to three weeks or more in vitro. FIG. 1C.

Step 4: Recovery and Injection. To recover the BAMs, the alginate microwell template was dissolved using a calcium chelator solution (such as sodium citrate or Ethylenediaminetetraacetic acid (EDTA)), typically 5% w/v sodium citrate in buffer solution (such as PBS or HEPES buffered saline) which recovers the BAMs into solution. The BAMs were then concentrated in solution by centrifugation or filtering and transferred to a syringe. The BAMs were injected in defined quantities (˜50-200 micro liters in mice) throughout the subcutaneous tissue of a patient (for example, BAMs can be distributed within the subcutaneous white adipose tissue). FIG. 1D.

Step 5: Integration and Vascularization of BAMs in vivo. After injection, the primitive blood vessel structures in each BAM integrated with each other and with the patient's blood vessels such that blood was rapidly perfused through the graft. This process ensured survival of the graft and establishment of the high vascular density required for optimal thermogenic function of the graft. The production of beta 3 adrenoreceptors on the BAMs (via in vitro adrenergic stimulation with beta 3 agonist CL316,243) allowed for integration with adrenergic neurons after implantation, enabling the in vivo stimulation and activation of the BAMs. FIG. 1E.

### Example 3

### Formation of Open Vascular Networks in BAMs Consisting of ASC and EC

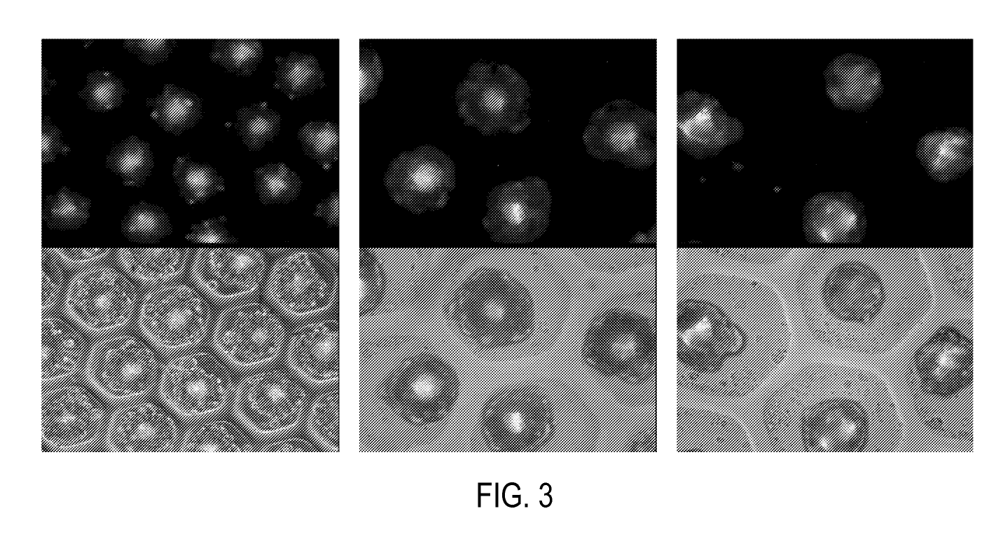

As can be seen in FIG. 3, BAMs consisting of ASC and EC formed open vascular networks after treatment with angiogenic factors (VEGF, bFGF). On the left, human adipose stem cells (ASC, unlabeled) and human GFP expressing endothelial cells (EC, bright white) were observed 1 day following seeding on the hydrogel microwell array. ECs were observed migrating to the center of the cellular aggregates. In the center of FIG. 3, ASC-EC aggregates were observed after several weeks in culture with factors promoting brown adipose differentiation. Lipid-containing ASC-derived cells were also observed around a solid core of EC. To the right, ASC-EC aggregates were further treated with angiogenic factors. ECs were observed to form primitive blood vessel structures with open lumens, with some branching structures visible.

### Example 4

### Demonstration of In vitro Differentiation and In vivo Integration of iBAMs

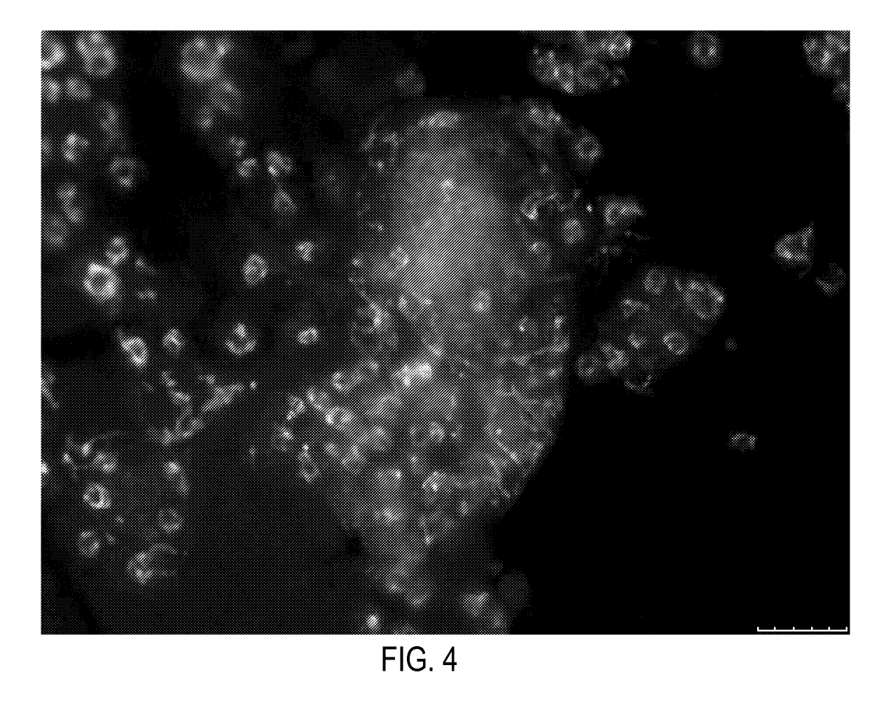

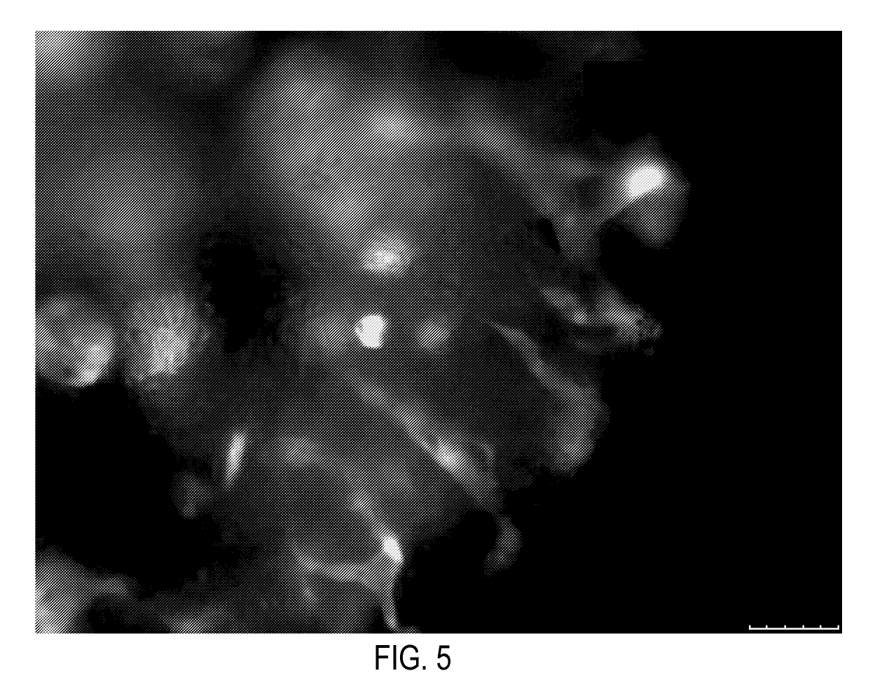

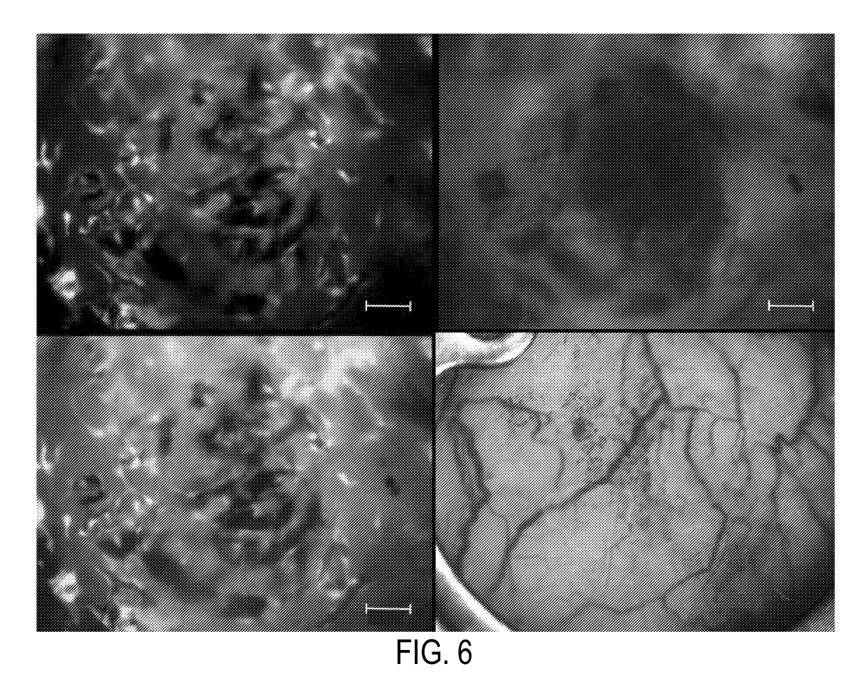

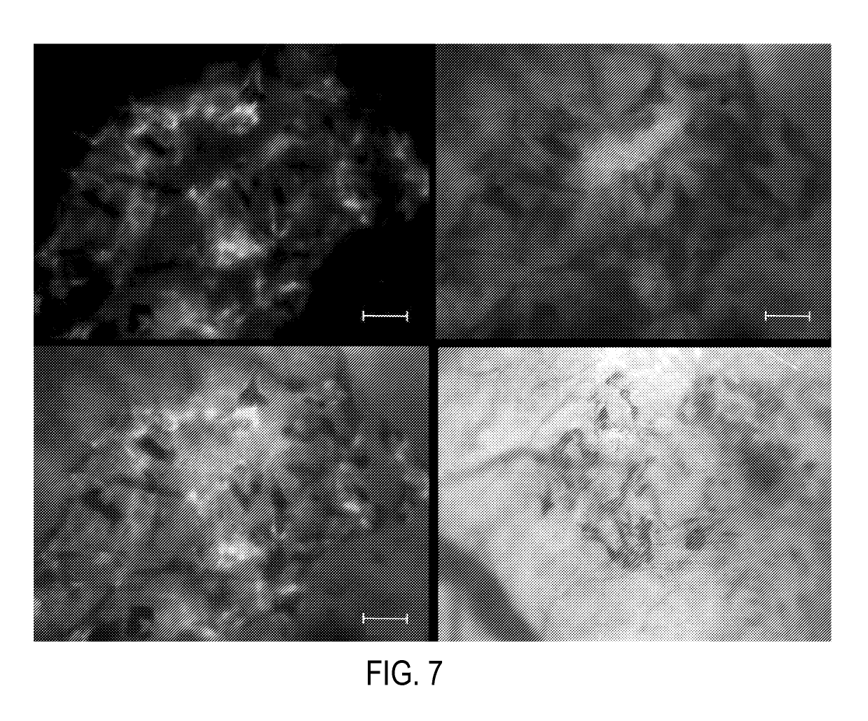

After implantation in SCID mice, human vessels in BAMS connected and merged with mouse vasculature and became perfused with blood (FIGS. 4-7). After in vitro assembly and culture of human BAMS, the BAMs were collected and injected into a dorsal skinfold window chamber in SCID mice. Clusters of BAMS injected in a SCID mouse are shown 48 h post implantation with bright white showing the GFP-expressing human endothelial cells. FIG. 4. Some branching EC structures with open lumens were visible. Some lipid droplets in the surrounding unlabeled differentiated ASC were also observed. FIG. 5. After 1 week in vivo, extensive vascular networks lined with human-derived (GFP expressing) EC were visibly filled with blood. In FIG. 6, the top left panel shows GFP fluorescence (human EC). The top right panel shows bright field (blood filled vessels appearing dark), and the bottom left panel is a merged fluorescent/bright field image. The bottom right panel is a color stereoscope image showing ectopic blood vessel formation by human EC in the mouse dorsal skinfold window chamber. After 12 days in vivo, as seen in FIG. 7, human vascular networks were observed and continued to grow, remodel and mature. Host blood vessels were also observed to grow and connect with human implant-derived vessels. The top left panel shows GFP fluorescence (human EC), the top right panel shows bright field (blood filled vessels appearing dark), and the bottom left panel is a merged fluorescent/bright field image. The bottom right panel is a color stereoscope image showing ectopic blood vessel formation by human EC in the mouse dorsal skinfold window chamber.

### Example 5

### ASC to BAT Differentiation

The ideal duration of differentiation of human adipose-derived stem cells (ASCs) was determined in vitro. Human ASCs were treated with a brown adipogenic cocktail as shown below in Table 2. Immunostaining and fluorescence microscopy were used to determine and quantify the present of brown adipose tissue functional markers. The human ASCs treated with the cocktail expressed functional markers of brown adipose thermogenesis, including uncoupled protein 1 (UCP1)—the mitochondrial membrane responsible for thermogenesis in BAT—and β3 adrenergic receptors (β3AR)—stimulated by SNS in native BAT to upregulate UCP1 via a cAMP pathway—which increased over several weeks with chronic exposure.

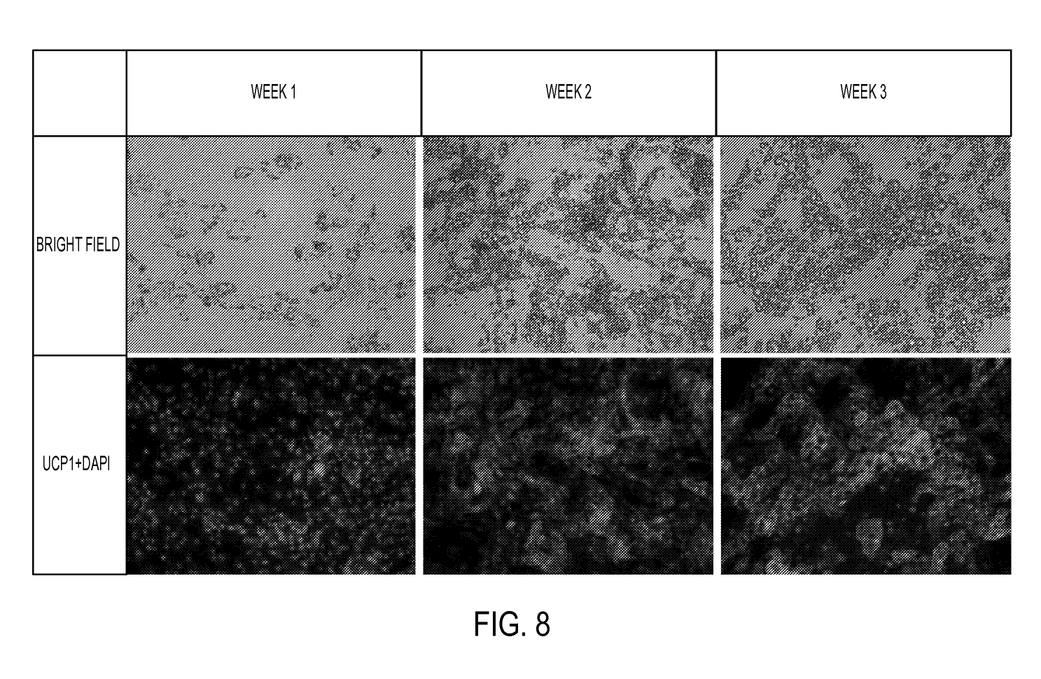

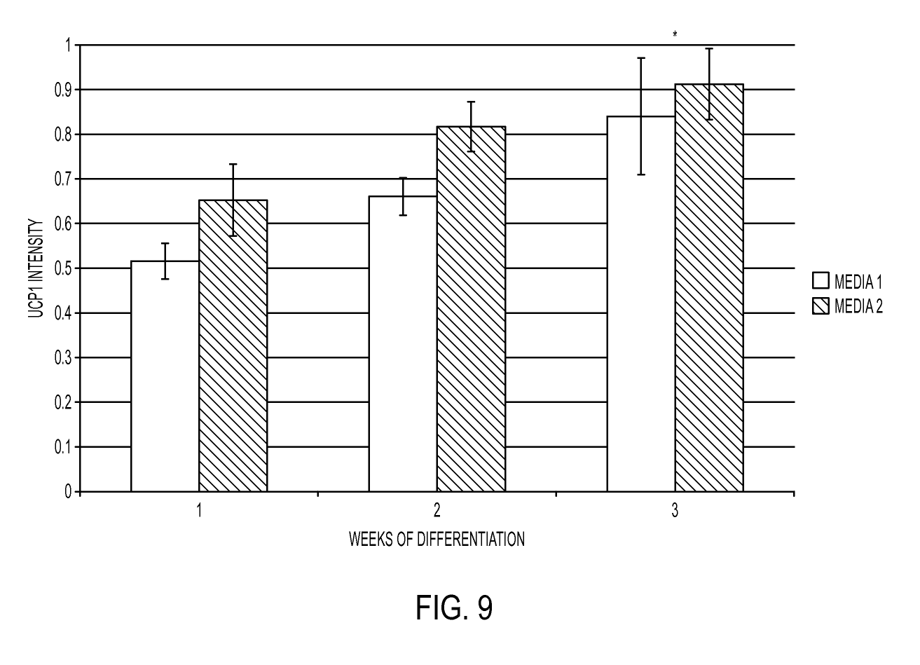

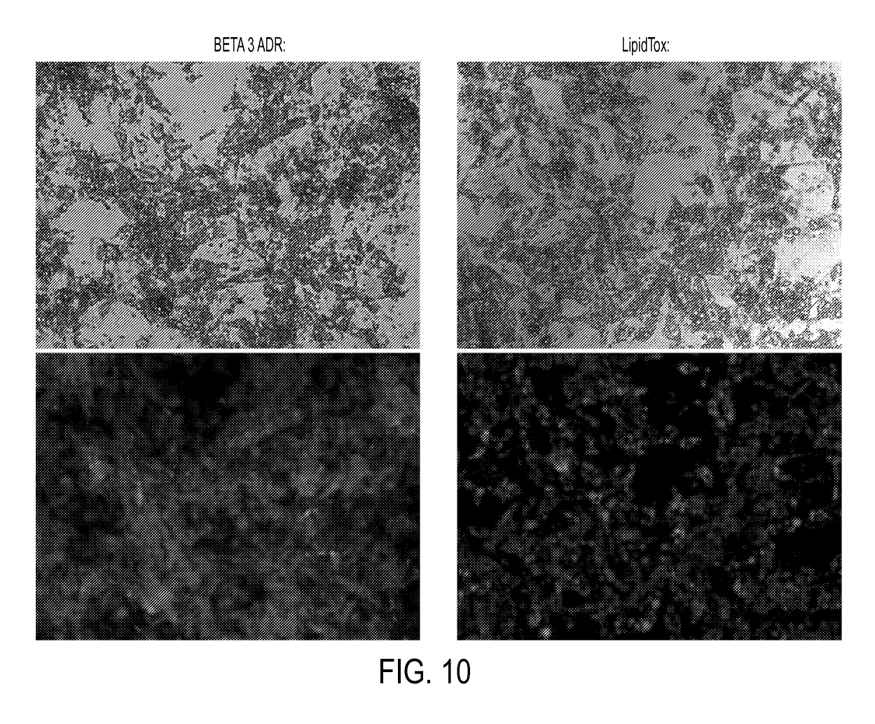

In FIG. 8, bright field and fluorescence images were taken of ASC cultured in brown adipogenic media for differentiation periods of 1, 2, and 3 weeks. UCP1 immunostaining (red) shows increasing amounts of UCP1 protein over the 3 weeks in culture. Quantification of UCP1 immunostaining over the course of 3 weeks of differentiation can be seen in FIG. 9. An increase in UCP1 immunostaining intensity occurred from 1-3 weeks' culture in brown adipogenic medium (Medium 1). Medium 2 was additionally supplemented with CL316,243. Finally, bright field (top) and fluorescence (bottom) images in FIG. 10 show ASC grown in brown adipogenic cocktail. In the left panel, positive immunostaining with anti-β3 adrenoreceptor antibodies indicates differentiated cells that express β3Ar. In the right panel, a fluorescent lipid stain highlights multilocular lipid droplets that are characteristic of brown adipose cells.

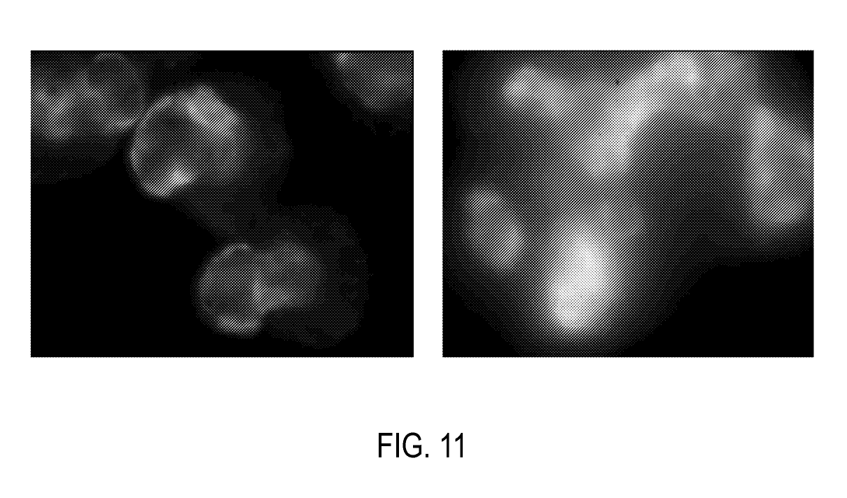

In the fluorescence images in FIG. 11, GFP-expressing human EC in four adjacent BAMs were observed to merge vascular structures after 24 h culture in media containing angiogenic factors (VEGF, bFGF).

### Creation of BAT Using Explanted White Adipose Tissue

### Example 6

### A Modified Approach to Creating Brown Adipose Tissue Involving Differentiation of Explanted White Adipose Tissue

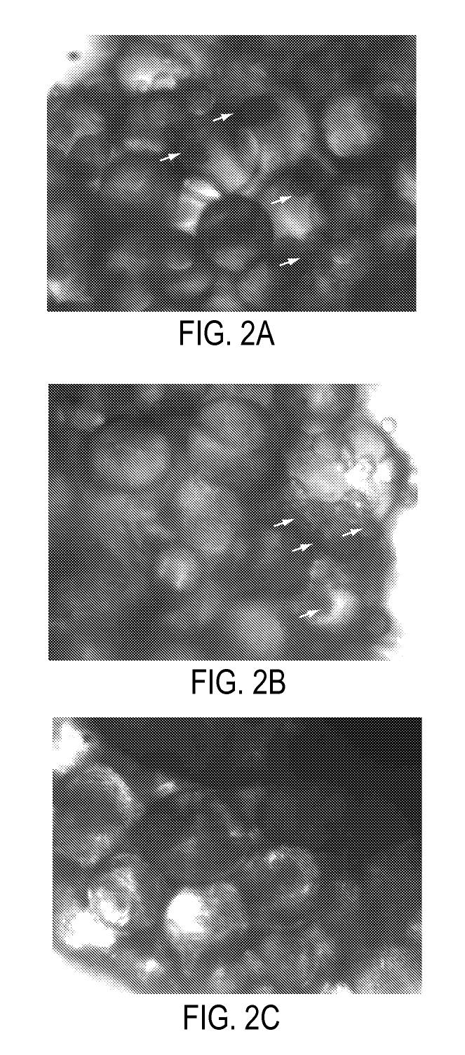

In some embodiments, brown adipose microtissues (BAMs) were produced by directly differentiating WAT fragments. In this approach, WAT was extracted from the host (such as by liposuction or surgical excision), and the tissue can be reduced to smaller fragments by mechanical means (such as by mincing or dicing). The WAT fragments were cultured in a bioreactor or culture vessel and exposed to factors that promote BAT differentiation, activation, and vascularization by cells present within the WAT fragments. A variety of bioreactor designs could be used, and include, but are not limited to, rotating wall vessels, perfusion bioreactors (e.g. fluidized tissue beds), tissue culture flasks, petri dishes, multiwell plates, spinner flasks, stir tank reactors, roller bottles, and gas permeable or non-gas permeable cell culture bags. In FIG. 2, images of explanted mouse white adipose tissue cultured in vitro in the presence or absence of brown adipogenic and angiogenic factors can be seen. In WAT cultured in brown adipogenic cocktail, small cells morphologically resemble brown adipocytes (containing multilocular lipid droplets) and were seen interspersed within large unilocular white adipocytes (arrows point to some BAT like cells). In panel A, the tissue was treated with a cocktail containing dexamethasone, indomethacin, insulin, isobutylmethylxanthine (IBMX), rosiglitazone, sodium ascorbate, triiodothyronine (T3), and CL316,243. In panel B, the tissue was treated with the same media as in A supplemented with additional angiogenic factors (VEGF and bFGF). In panel C, control growth media was used, and BAT-like cells were not observed. All images were taken after 17 days of culture in each condition.

### Example 7

### Ex vivo Browning of WAT Fragments to Convert WAT to BAT

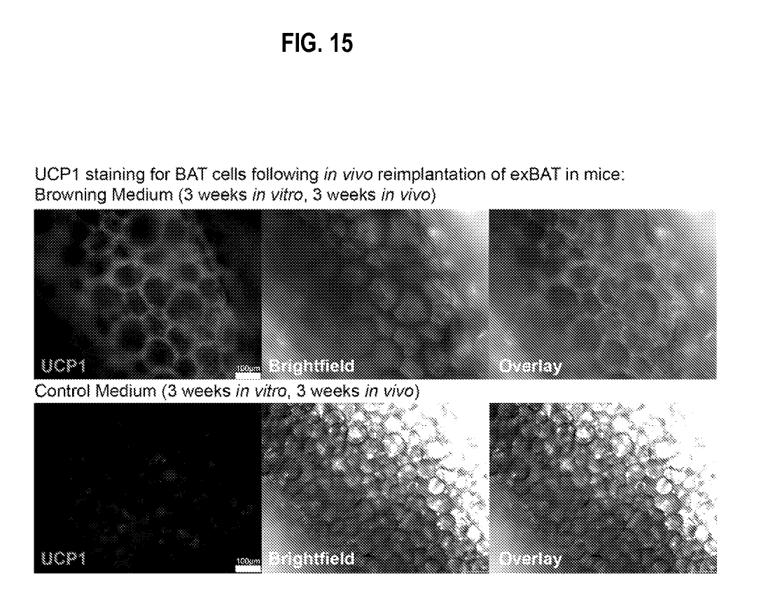

In FIG. 15A small amounts (˜100 mg) of subcutaneous inguinal WAT were surgically extracted and minced into ˜2 mm size pieces from 30-week-old DIO mice, and then transferred to 24 well plates in control media or media containing browning and angiogenic factors. The optimal media composition and culture time were determined for robust development of UCP1-expressing BAT cells and angiogenesis in the WAT explants. Browning of isolated WAT tissue fragments was achieved after culturing for up to one month. After 10 days in culture, development of clusters of UCP1 expressing cells containing multiple small lipid vacuoles, characteristic of BAT cells, within WAT using browning conditions was observed. Whole tissue fragments were immunostained for UCP1 and imaged on an epifluorescence microscope. Several z planes are shown to show that BAT-like cells develop throughout the WAT fragments. In FIG. 15B, control conditions without browning factors show no UCP1 expression or development of small multilocular adipocytes. The persistence of viable vascular structures was also observed within the tissue as indicated by live cell staining of calcein AM. Reimplantation procedures were performed in a small number of mice using “subtherapeutic” doses of ThermoGraft, and so far observed no adverse effects.

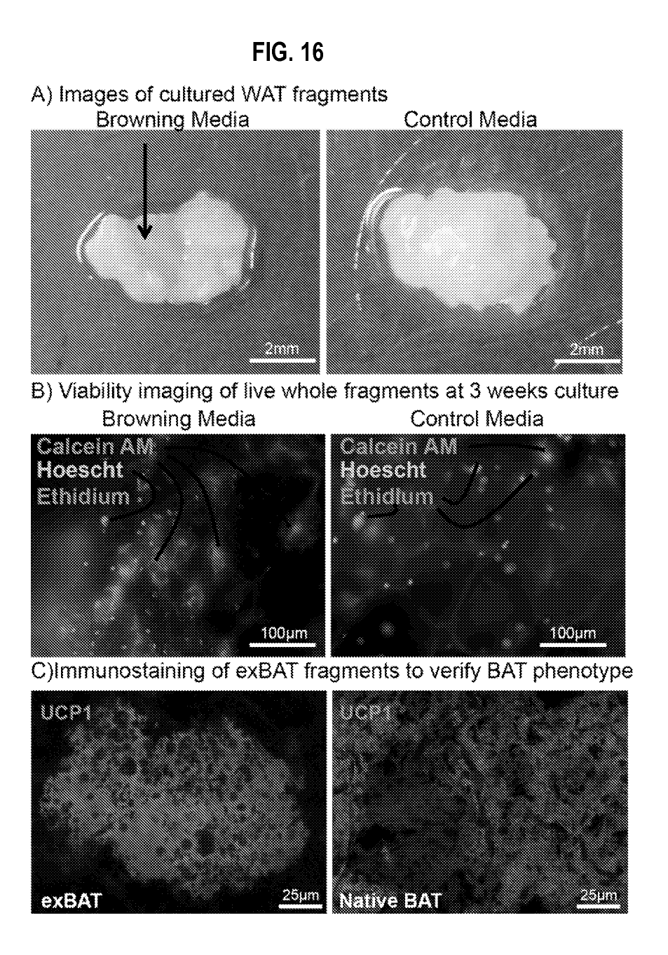

In FIG. 16A, images of cultured WAT fragments show browning after addition of browning media vs. control media. FIG. 16B shows viability imaging of live whole fragments at 3 weeks' culture. WAT fragments cultured for 21 days ex vivo were stained with Calcein AM to label live cells and Hoescht to label nuclei. FIG. 16C includes images of exBAT fragments immunostained to verify BAT phenotype as a high magnification confocal slice showing UCP1 immunostaining of ThermoGraft (10 days' browning) and native BAT.

### Example 8

### Devices for Pre-Assembly of Multiple BAMs in Defined Shapes Prior to Injection

A method to assemble multiple vascularized microtissues together to form larger tissues with extensively connected vascular networks was developed. When microtissues were placed together in a medium containing angiogenic factors, blood vessel structures in each microtissue grew and connected with adjacent vessels in vitro. Development of more extensively connected networks prior to implantation may accelerate perfusion of the graft with blood, since fewer connections need to be made following implantation.

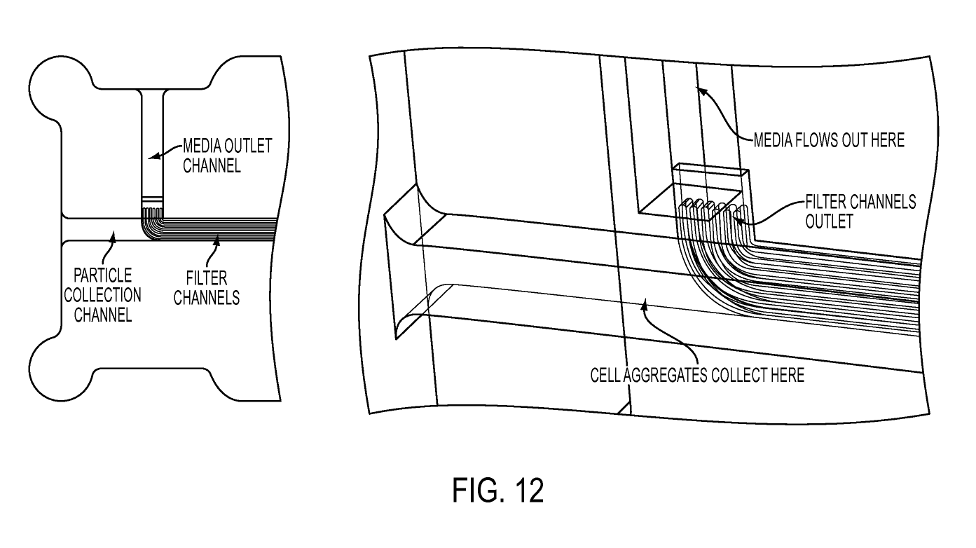

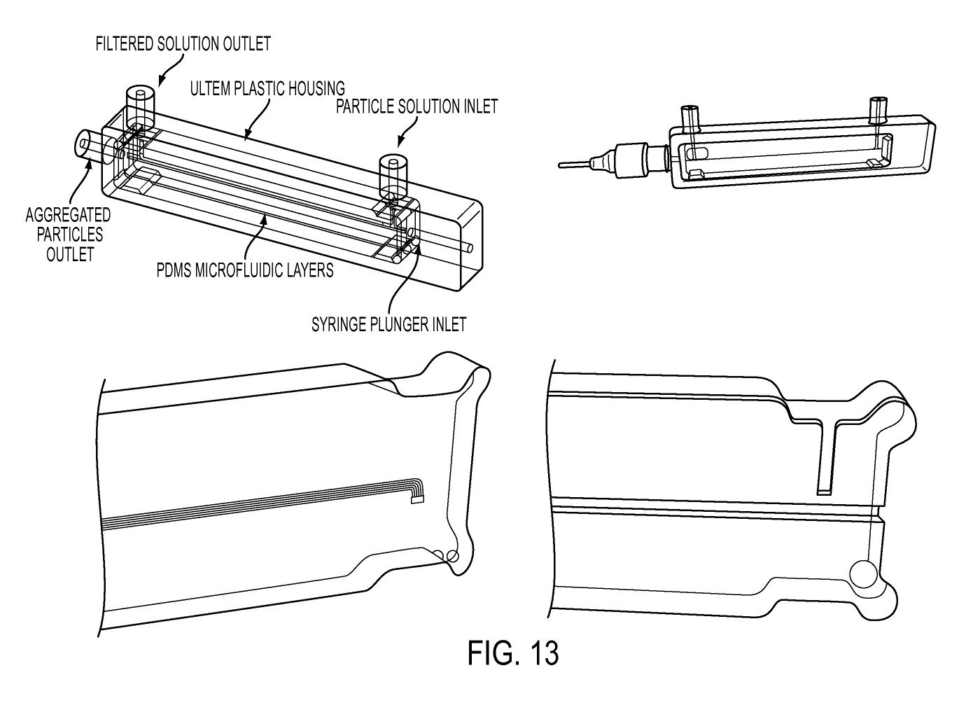

Aggregates of multiple microtis sues were formed in different shapes by collecting them within microwells or microchannels. For example, thin fibers were made by collecting microtissues within microchannels. A fiber geometry is advantageous for in vitro culture since diffusion distances remain small, and the fiber can still be injected through a small diameter needle. A syringe device can form fibers of BAMs within a channel, permit media flow around the fiber to allow for extended culture, and allow the fibers to be directly injected to the patient (FIGS. 12 and 13).

### Example 9

### Assessment of Tissue Viability and Extent of Browning Using Mouse Adipose Fragment

A 8 μM Calcein solution in serum-free medium was prepared by adding 2 μL/mL of 4 mM calcein stock solution. A 2 μM Ethidium solution was prepared in the calcein solution by adding 1 μL/mL of 2 mM ethidium stock and to Calcein solution. 20 μg/mL Hoescht solution was then prepared in the calcein solution by adding 2 μL/mL of 10 mg/mL hoescht stock and to Calcein solution. The mouse adipose tissue fragments were incubated in Calcein/ethidium solution for lhr in the 5% CO₂ 37° C. incubator. The mouse adipose fragments were washed in PBS twice for 5 min and then incubated in Calcein/hoescht solution for lhr in the 5% CO₂ 37° C. incubator. The mouse adipose fragments were then washed again in PBS twice for 5 min. The fragments were placed on a slide, coverslip added so as not to crush the tissue, and imaged using a fluorescence microscope.

After dissection, mouse adipose tissue samples were placed into 4% PFA at 4° C. overnight. On day 1, the 4% PFA-fixed mouse adipose tissues were immersed in Petri dishes filled with 1× PBS. The mouse adipose tissues were cut into thin slices, e.g., 2 mm×2 mm and washed in a well plate with 1× PBS for an hour on a rocking board to remove PFA. The mouse adipose tissues were digested by incubating with proteinase K at room temperature for 5min. Proteinase K solution was made by adding 0.03152 g of trisma hydrochloride in 20 ml of DI water. 20 ul of proteinase K was added into the 20 ml solution above. The samples were washed with PBS for a few seconds and then the adipose tissues were incubated with 100% methanol (0.75 ml) at room temperature for 30min in a chemical fume hood. The adipose tissue was washed thoroughly with PBS for an hour on a rocking board (×3 times) for a total of 3 hr. After 3 hr, the PBS was removed and the adipose tissue was incubated in 3% blocking buffer at 4° C. for 12-24 hr on a rocking board to block nonspecific binding sites. The block buffer was made by adding 1.5 g of milk powder into 50 ml solution, which can be made by adding 1.5 ml of triton into 50 ml PBS.

On day 2, the blocking buffer was removed by washing with 3% triton 100× for 15 min at 4° C. Blocking buffer was also made for primary antibody UCP-1. 3% triton solution was removed and the adipose tissue was incubated with primary antibodies for 24 hr at 4° C. on a rocking board. The antibody stock was diluted to 1:200 in blocking buffer.

On day 3, the primary antibody solution was removed, and the samples were rinsed in PBS for a few seconds. The adipose tissue was washed with 3% triton for 1.5 hr on a rocking board at 4° C. The 3% triton solution was removed. The adipose tissues were then incubated with 3% blocking buffer for 1.5 hr at 4° C. on a rocking board. The adipose tissues were incubated with secondary antibody diluted in 3% blocking buffer for 2 hr at room temperature on a rocking board. The secondary antibody (e.g., Alexa fluor 555 goat anti rabbit Ab) was diluted in 1:400 in blocking buffer. The tissues were rinsed in 3% triton solution overnight at 4° C. on a rocking board.

On day 4, the adipose tissue were washed and imaged using a whole mount adipose tissue stain. The fixed tissue was cut into small pieces: ˜2 mm×2 mm and washed for an hour in PBS to remove PFA. The tissue was then digested in 20 μg/ml proteinase K at RT for 5 min to break down ECM. Next, the tissue was washed in PBS at RT for a few seconds to remove Proteinase K solution. The PBS was removed and then the tissue was incubated in Methanol at RT for 30 min to permeabilize the tissue. Methanol was removed and the tissue washed 3× at RT for 0.5 hrs (1.5 hr total) in PBS. PBS was removed and the tissue was incubated in in blocking buffer for 2 hr at 4° C. The blocking buffer was removed by rinsing for 15 min in 3% triton 100× at 4° C. The 3% triton solution was removed and primary antibody solution added (dilute antibody stock 1/200 in blocking buffer). Then, the tissue was incubated in primary antibody for 12 hrs at 4° C. A day later, the primary antibody solution was removed and tissue rinsed in PBS. The tissue was then washed in 3% triton twice for lhr. The triton solution was removed and the tissue incubated in blocking buffer for 2 hr at 4° C. The tissue was then incubated in secondary antibody diluted 1/400 in blocking buffer for 1 hr at 4° C. The tissue was then rinsed in 3% triton solution twice for lhr at 4° C. and imaged at the end of day 2.

### Example 10

### Optimization of Browning Conditions for Ex vivo Browning of Human WAT

It is possible to optimize browning conditions. In certain embodiments, we arel implementing Design-of-Experiments (DOE) “lean” development methodology to optimize the browning media and culture protocol for ex vivo browning of human WAT. DOE is a statistical method to determine the sensitivity of process outputs to varying process parameters, and is a commonly used approach for optimizing industrial cell culture bioprocesses through minimal experimentation. We will vary key browning and environmental factors (see Table 1), and measure both functional and phenotypic/morphologic properties of the tissue over time to determine the optimal factor concentrations and environmental conditions for up to 1 month ex vivo. In certain embodiments, we will first experiment with a minimal set of factors (indicated by asterisks in Table 1), and expand to additional factors if browning or angiogenesis is not adequate across a spectrum of patients.

TABLE 1
List of factors to be investigated for optimizing ex vivo browning

| Tissue | Browning | Vascularization | Environment |
|---|---|---|---|
| maintenance: | factors: | factors: | factors: |
| Insulin* | Temperature | Vascular | Flow rate* |
| (0-1 μM) | (15-37 C.)* | endothelial | (1-100 mL/min) |
| Hydrocortisone* | Norepinephrine* | growth factor | Media |
| (0-10 nM) | (0-10 μM) | (VEGF) | exchange rate* |
| Sodium | Thyroxine (T3) | (0-50 nM) | (10-500%/day, |
| ascorbate* | (0-1 μM) | Basic fibroblast | i.e. up to 5 |
| (0-25 μg/mL) | Retinoic Acid | growth factor | chamber volume |
| | (0-10 μM) | (bFGF) ) | media changes |
| | Orexin(0-10 μM) | (0-50 nM) | daily) |
| | Rosiglitizone | | % CO₂* (0-10%) |
| | (0-10 μM) | | % O₂ (10-30%) |

*Primary factors of interest for minimum factor browing conditions

In certain embodiments, we will use miniaturized high-throughput versions of our bioreactor to culture human WAT under a large number of conditions. We can readily obtain 20 mL of fat from autologous fat transfer patients, and use 0.5-1 mL of WAT per chamber. Only a few milligrams of tissue are required for metabolic and histological assays, so sampling from individual chambers can be performed over time to efficiently examine the influence of culture time on browning in each condition.

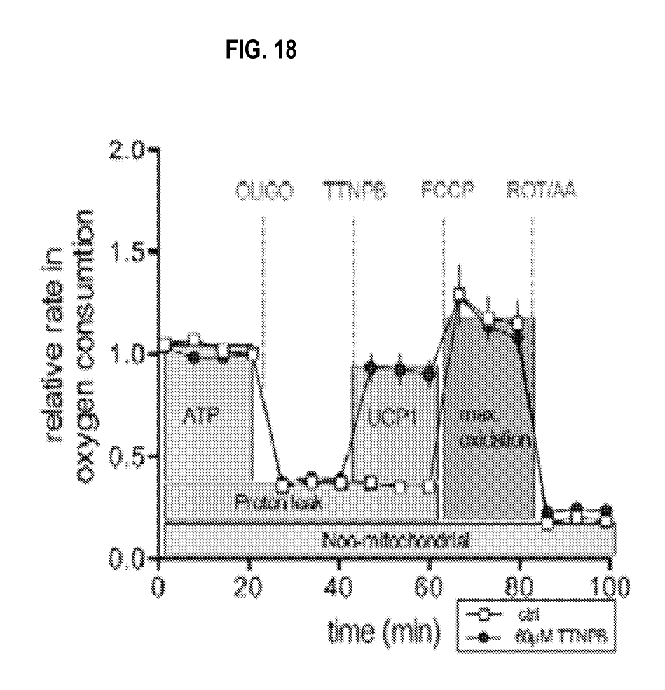

In certain embodiments, we will perform whole-mount immunostaining for UCP1 and CD31 (blood vessels) (as shown in FIG. 16), and quantify the number of UCP1-positive cells per mL tissue and vascular density by microscopy. We will measure functional metabolic properties (basal, uncoupled, and maximal respiration) using the Seahorse XF^{e}24 system (FIG. 18). Measurements are made immediately upon tissue collection, and at days 1-3, 5, 7, 10, 14, 21, and 28 of ex vivo culture in the various conditions.

### Example 11

### Example for Ex vivo Browning Process

In this example, a mouse model was developed to test if whole WAT fragments could be converted to BAT ex vivo, and whether the BAT phenotype could persist after long-term implantation (FIG. 19B). A piece (˜0.5 mL) of subcutaneous WAT was excised from the left inguinal depot (located along the rear flank of the mouse above the hindlimb), and then minced the tissue into fragments of approximately 2 to 5 mm in diameter. The fragments were suspended in either browning media or control media without browning factors. A cocktail of browning factors including rosiglitazone (ppary agonist), isobutylmethylxanthene (IBMX, phosphodiesterase inhibitor), T3 (thyroid hormone), indomethacin (COX inhibitor), CL316,243 (β3 adrenoreceptor agonist), and vascular endothelial growth factor (VEGF) were used. In an aspect, control media was prepared by adding 10% fetal bovine serum (FBS), 1% Penicillin Streptomycin, 20 mM HEPES, 50 μg/mL sodium ascorbate, 1 μM insulin into Dulbecco's Modified Eagle Medium (DMEM). In another aspect, The browning media was prepared by adding 1 μM Dexamethasone, 500 μM Isobutylmethylxanthine, 50 μM Indomethacin, 1 μM Rosiglitazone and 1 μM CL316243, and 250 nM triiodothyronine (T3), and 25 ng/mL VEGF into control media.

In an aspect, tissue fragments cultured in control media retained a WAT-like appearance, and it was observed that tissues cultured in browning media exhibit a brown color consistent with BAT which contains a high density of iron-rich mitochondria (FIG. 19C). Live-cell staining on whole tissue fragments was performed in the presence of browning media (FIG. 19D). Initially, the WAT fragments displayed cytoplasmic and mitochondrial staining around large lipid droplets, as well as in branching vascular structures (FIG. 19D, left panels). After 1 week in browning media, tissues displayed higher cell density and more numerous, smaller lipid droplets, consistent with formation of BAT-like tissue (FIG. 19D right panels).

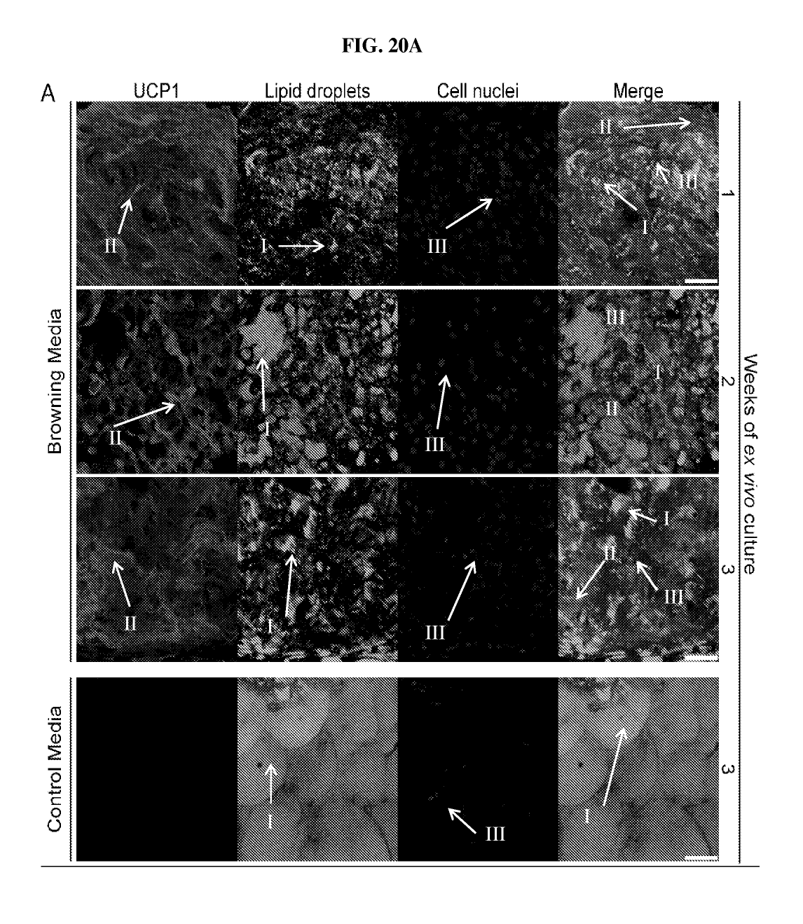

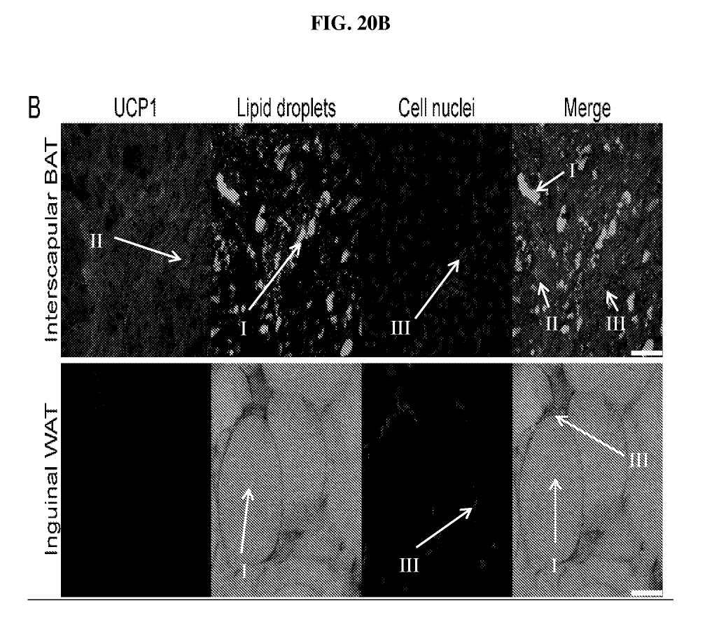

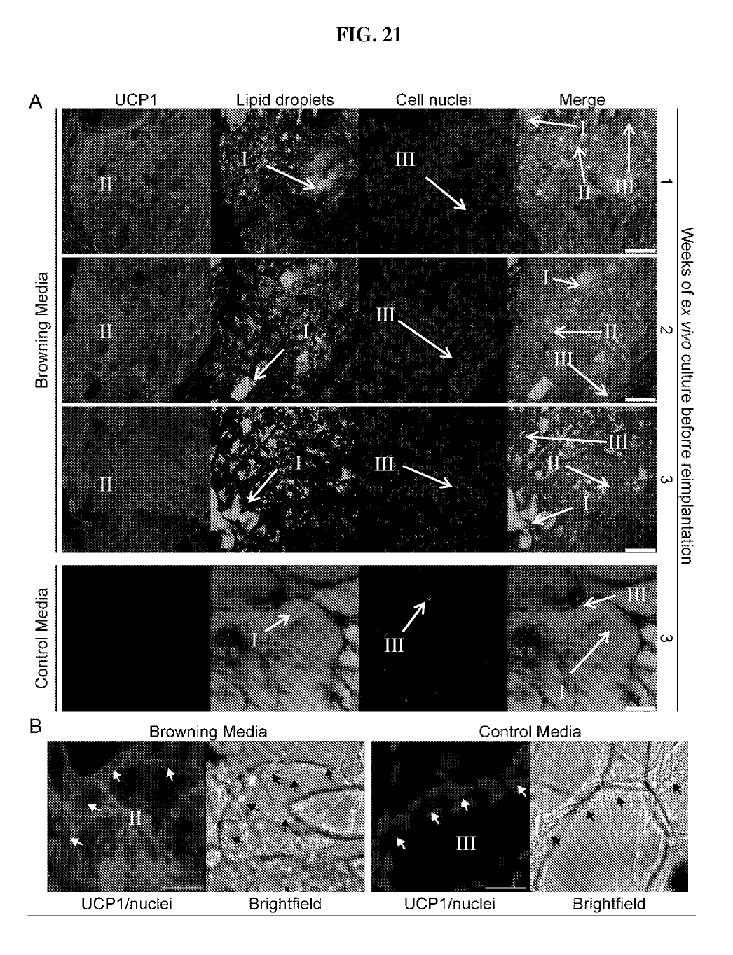

The conversion of WAT to BAT through fluorescence microscopy was qualified and quantitative image analysis of tissue fragments labeled for UCP1, lipids, and cell nuclei (FIGS. 20-22). Immunostaining on whole tissue fragments with anti-UCP1 antibodies was tested, and counterstained lipids with Lipidtox and cell nuclei with Hoescht or Sytox. It was observed that tissues cultured in browning media for 1-3 weeks exhibited high UCP1 signal, numerous small lipid droplets, and a high cell density (FIG. 20A top rows), appearing similar to native interscapular BAT (FIG. 20B top row). By contrast, tissues cultured in control media (FIG. 20A, bottom row) appeared similar to inguinal WAT (FIG. 20B bottom row), exhibiting low UCP1 signal, large lipid droplets, and lower cell density. Thus, ex vivo browning exhibited hallmarks of the native-like browning process in vivo.

### Example 12

### Testing of Persistence of BAT-Like Phenotype in Mice

Tissues cultured in browning media were evaluated 8 weeks after reimplantation. After the ex vivo culture period, portions of tissues were subcutaneously reimplanted on the right inguinal WAT depot.

It was observed that tissue fragments fused into a vascularized fat pad was distinguishable from the underlying inguinal WAT after 8 weeks (FIG. 19C). The whole tissue fragment for UCP1 were immunostained and counterstained to label lipid droplets and cell nuclei. Tissues cultured in browning media exhibited high levels of UCP1 signal, numerous small lipid droplets, and a high density of cell nuclei (FIG. 21A top rows). Tissues cultured in control media prior to reimplantation retained a WAT-like appearance (FIG. 21A bottom row). Functional blood vessels were observed within the reimplanted tissues, as indicated by red blood cell-filled vessels that were visible in transmitted light images (FIG. 19B). Larger blood-filled vessels entering the regrafted tissue were also visible by eye.

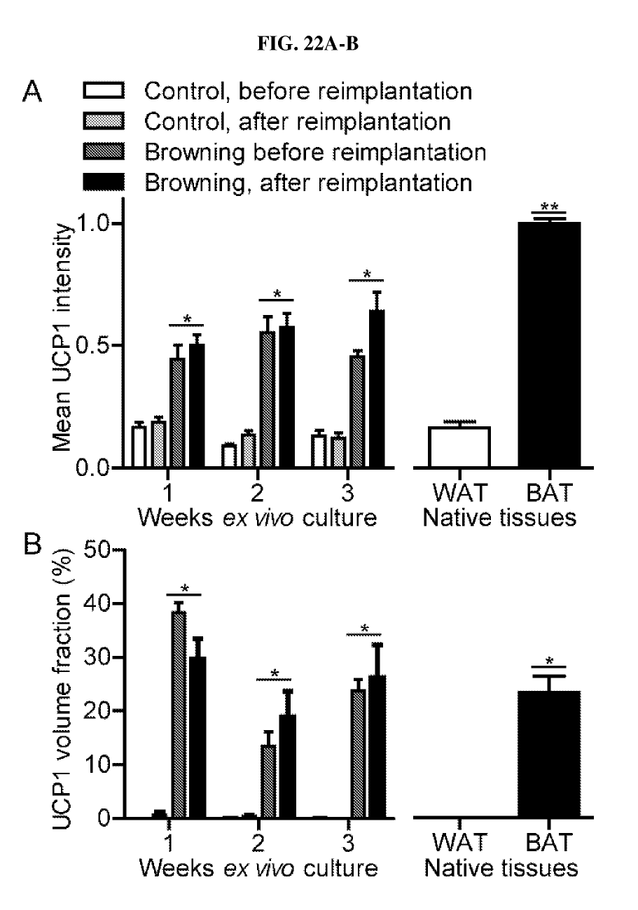

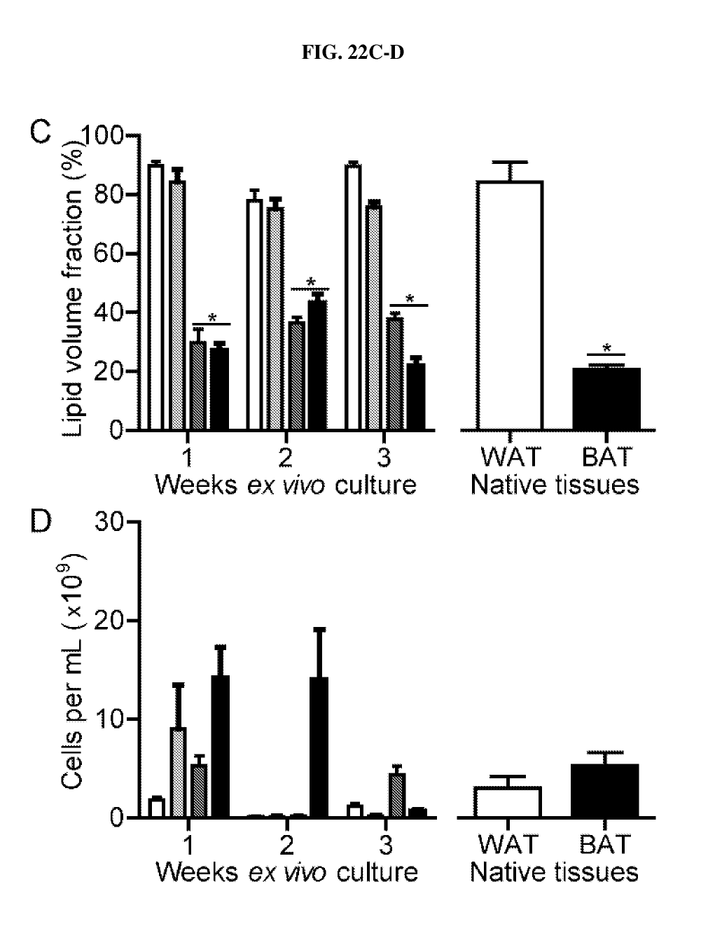

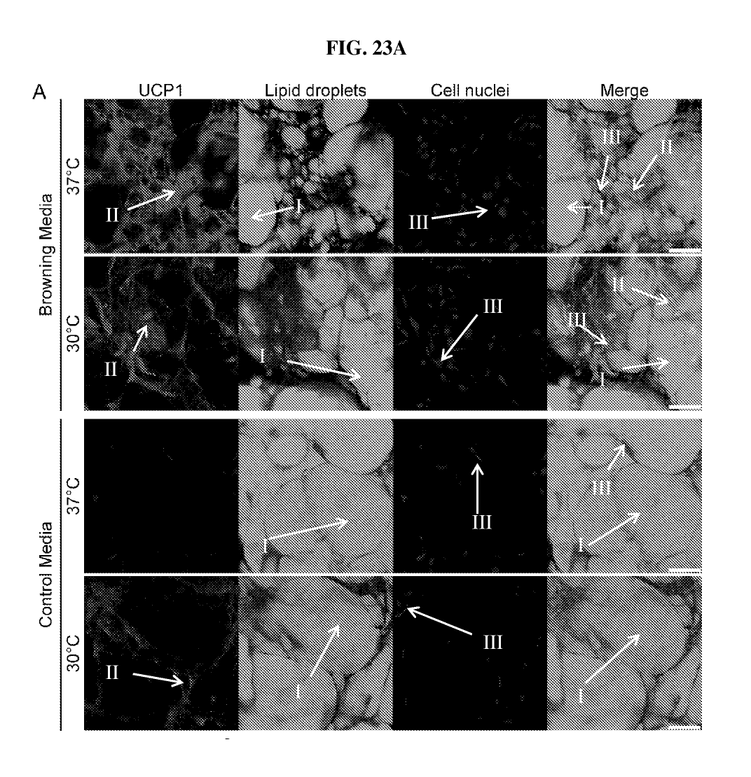

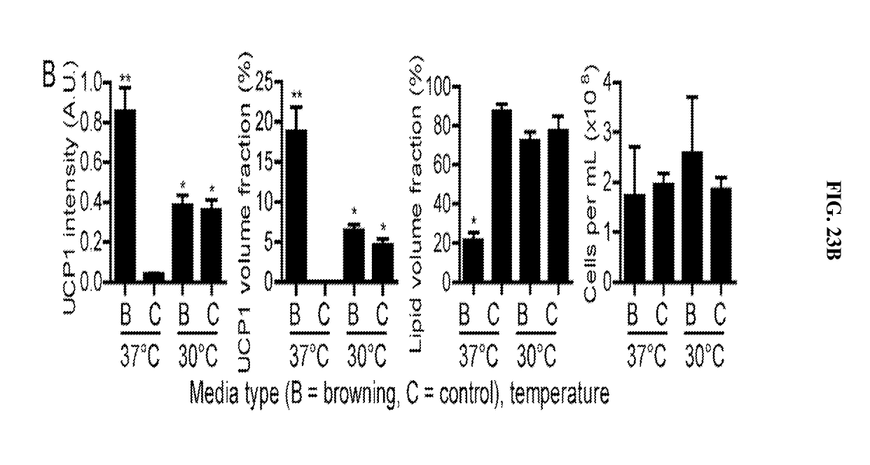

Using widefield epifluorescence images of large areas of tissue fragments, UCP1 immunostaining intensity was evaluated. UCP1 intensity levels were significantly higher (p<0.001) for tissues cultured in browning media compared to control media at each time point, both before and after reimplantation, as determined by two-way ANOVA and Bonferroni post hoc tests as, for example, indicated by single asterisks in FIG. 22 B. The culture time did not significantly impact UCP1 intensity levels for tissues cultured in both media types. The UCP1 intensity levels of tissues cultured in browning media reached approximately 40-70% of the intensity of interscapular BAT, and did not appear as UCP1-dense in low magnification images used for quantification. As a control, the UCP1 intensity levels of inguinal WAT tissue was similar to tissues cultured in control media and significantly lower than tissues cultured in browning media and inguinal BAT.

3D confocal image stacks of tissues through segmentation of UCP1, lipid, and nuclear staining were also evaluated. See, for example, FIG. 22 B-D. UCP1 fraction measurements were statistically similar between tissues cultured in browning media and interscapular BAT, while the UCP1 fraction for tissues cultured in control media and inguinal WAT were extremely low (FIG. 22B). Quantification of lipid fraction readily distinguished interscapular BAT from inguinal WAT (FIG. 22C). The lipid fraction of tissues cultured in browning media was statistically similar to that of interscapular BAT, while the lipid fraction of tissues cultured in control media was similar to that of inguinal WAT. The duration of culture did not have a significant effect on the lipid fraction for either culture media.

### Example 13

### exBAT Browning Conditions on Human Subcutaneous WAT

The conversion of human subcutaneous WAT to BAT-like tissue by ex vivo browning (FIG. 23) was investigated. For these studies, excess subcutaneous WAT donated by two patients who underwent autologous fat grafting procedures under an IRB-approved protocol was collected. Tissues were cultured in the same media and browning factors as mouse tissues, except that VEGF was omitted. It was observed that human WAT developed UCP1 expression and smaller lipid droplets when cultured in browning media for 1 week (FIG. 23A top row), while tissues cultured in control media remained WAT-like (FIG. 23A second row), similarly to mouse tissues. In addition to using browning factors, the use of cold stimulation to induce browning by culturing some tissues at 30° C. was also investigated. It was found that culture at reduced temperature increased UCP1 expression in the absence of browning factors (FIG. 23A bottom row), but decreased UCP1 expression relative to 37° C. culture when tissues were cultured with browning factors (FIG. 23A third row). The visible changes were reflected in quantitative assessment of UCP1 intensity and UCP1 fraction, which showed statistically significant differences between browning media and controls at 37° C. but not 30° C. (FIG. 23B).

In the specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

One of ordinary skill in the art can make many variations and modifications to the above-described embodiments of the invention without departing from the spirit or scope of the appended claims. Accordingly, all such variations and modifications are within the scope of the appended claims.

1. A method comprising:
(a) harvesting subcutaneous white adipose tissue fragments from a subject;
(b) transferring the white adipose tissue fragments into a bioreactor;
(c) culturing the white adipose tissue fragments in the bioreactor wherein culturing occurs
(i) in the presence of media comprising browning factors, or
(ii) in the presence of cold temperature, wherein said cold temperature is in the range of from about 15° C. to 35° C., or
(iii) in the presence of a combination of both media comprising browning factors and cold temperature; wherein said cold temperature is in the range of from about 15° C. to 35° C.;
thereby promoting conversion of white adipose tissue fragments to brown adipose tissue fragments.

2. The method of claim 1 wherein the browning factors are selected from the group consisting of: insulin, norepinephrine, and hydrocortisone, Dexamethasone, Indomethacin, Isobutylmethylxanthine (IBMX), Rosiglitazone, Sodium Ascorbate, Triiodothyronine (T3), CL316,243, retinoic acid, vascular endothelial growth factor (VEGF), basic fibroblast growth factor (bFGF), fibroblast growth factor 21 (FGF21), bone morphogenetic protein 7 (BMP7) Orexin, irisin, Meteorin-like, β-Aminoisobutyric acid, brain derived neurotrophic factor (BDNF), TLQP-21, leptin, capsaicin, fucoxanthin, 2-hydroxyoleic acid, conjugated linoleic acid, Bofutsushosan, Resveratrol, beta adrenergic agonists, prostaglandins, peroxisome proliferator-activated receptor gamma (PPARγ) ligands, peroxisome proliferator-activated receptor alpha (PPARα) ligands, retinoids, thyroid hormones, AMP-activated protein kinase (AMPK) activators, n-3 fatty acids of marine origin, scallop shell powder, and salmon protein hydrolysate and analogs thereof.

3. The method of claim 2 wherein the browning factors are selected from the group consisting of 0-10 μM norepinephrine, 0-1 μM.insulin, and 0-10 nM hydrocortisone.

4. The method of claim 3 wherein the cold temperature is in the range of from about 25° C. to 35° C.

5. The method of claim 4 wherein the temperature is about 30° C.

6. The method of claim 5 wherein the therapeutically effective amount is in a range from about 0.02-20 kilograms.

7. A method of treatment for a metabolic disorder, comprising:
(a) harvesting subcutaneous white adipose tissue fragments from a subject;
(b) transferring the white adipose tissue fragments into a bioreactor;
(c) culturing the white adipose tissue fragments in the bioreactor wherein culturing occurs
(i) in the presence of media comprising browning factors, or
(ii) in the presence of cold temperature, wherein said cold temperature is in the range of from about 15° C. to 35° C.; or
(iii) in the presence of a combination of both media comprising browning factors and cold temperature; wherein said cold temperature is in the range of from about 15° C. to 35° C.;
thereby promoting conversion of white adipose tissue fragments to brown adipose tissue fragments;
(d) recovering the brown adipose tissue fragments from the bioreactor; and
(e) administering a therapeutically effective amount of the isolated brown adipose tissue fragments to the subject.

8. The method of claim 7 wherein the therapeutically effective amount is in a range from about 0.02-20 kilograms.

9. The method of claim 8 wherein the browning factors are selected from the group consisting of: insulin, hydrocortisone, and norepinephrine, Dexamethasone, Indomethacin, Isobutylmethylxanthine (IBMX), Rosiglitazone, Sodium Ascorbate, Triiodothyronine (T3), CL316,243, retinoic acid, vascular endothelial growth factor (VEGF), basic fibroblast growth factor (bFGF), fibroblast growth factor 21 (FGF21), bone morphogenetic protein 7 (BMP7) Orexin, irisin, Meteorin-like, (3-Aminoisobutyric acid, brain derived neurotrophic factor (BDNF), TLQP-21, leptin, capsaicin, fucoxanthin, 2-hydroxyoleic acid, conjugated linoleic acid, Bofutsushosan, Resveratrol, beta adrenergic agonists, prostaglandins, peroxisome proliferator-activated receptor gamma (PPARγ) ligands, peroxisome proliferator-activated receptor alpha (PPARα) ligands, retinoids, thyroid hormones, AMP-activated protein kinase (AMPK) activators, n-3 fatty acids of marine origin, scallop shell powder, and salmon protein hydrolysate and analogs thereof.

10. The method of claim 9 wherein the browning factors are selected from the group consisting of 0-10 μM norepinephrine, 0-1 μM.insulin, and 0-10 nM hydrocortisone.

11. The method of claim 10 wherein the cold temperature is in the range of from about 25° C. to 35° C.

12. The method of claim 11 wherein the metabolic disorder is selected from the group consisting of: obesity, overweight, type 2 diabetes, metabolic syndrome, impaired glucose tolerance, insulin-resistance, dyslipidemia, cardiovascular disease, and hypertension.

13. A pharmaceutical composition comprising therapeutically effective amounts of brown adipose tissue fragments made by the methods of claim 1.

14. A kit comprising the pharmaceutical composition of claim 13.

15. A browning medium comprising factors selected from the group consisting of: insulin, hydrocortisone, and norepinephrine, dexamethasone, indomethacin, isobutylmethylxanthine (IBMX), rosiglitazone, sodium ascorbate, triiodothyronine (T3), CL316,243, retinoic acid, vascular endothelial growth factor (VEGF), basic fibroblast growth factor (bFGF), fibroblast growth factor 21 (FGF21), bone morphogenetic protein 7 (BMP7), orexin, irisin, meteorin-like, β-aminoisobutyric acid, brain-derived neurotrophic factor (BDNF), TLQP-21, leptin, capsaicin, fucoxanthin, 2-hydroxyoleic acid, conjugated linoleic acid, bofutsushosan, resveratrol, beta adrenergic agonists, prostaglandins, peroxisome proliferator-activated receptor gamma (PPARγ) ligands, peroxisome proliferator-activated receptor alpha (PPARα) ligands, retinoids, thyroid hormones, AMP-activated protein kinase (AMPK) activators, n-3 fatty acids of marine origin, scallop shell powder, and salmon protein hydrolysate and analogs thereof.

16. An apparatus comprising:
a gas permeable membrane configured to enclose, at least in part, a culture chamber;
a first port in fluid communication with the culture chamber;
a different second port in fluid communication with the culture chamber;
a tissue access port configured to pass a tissue fragment from about 1 millimeter in size to about 10 millimeters in size into and out of the culture chamber.

17. An apparatus as recited in claim 16, wherein
the first port is configured to be connected to an external supply of a fluid medium to allow flow of the fluid medium into the culture chamber; and
the second port is configured to pass fluid out of the culture chamber.

18. An apparatus as recited in claim 17, wherein
the apparatus further comprises a semi-permeable membrane separating the tissue access port from the second port; and
the semi-permeable membrane is configured to pass a waste product from the tissue fragment and not to pass the tissue fragment.

19. An apparatus as recited in claim 18, further comprising a rigid housing configured to hold the gas permeable membrane in a predetermined shape when the culture chamber is filled with a fluid, wherein the housing includes a vent configured to allow gas outside the housing to contact the gas permeable membrane.

20. An apparatus as recited in claim 19, wherein the apparatus is a configured for single use.

21-76. (canceled)
